(19) United States
(12) Patent Application Publication
(10) Pub. No.: US 2023/0301559 A1
(43) Pub. Date: Sep. 28, 2023

(54) NONINVASIVE BLOOD GLUCOSE MEASUREMENT APPARATUS AND METHOD USING MULTIPLE SENSORS

(71) Applicant: Electronics and Telecommunications Research Institute, Daejeon (KR)

(72) Inventors: Jong Deog Kim, Daejeon (KR); Bong Kyu Kim, Daejeon (KR); CHUL HUH, Daejeon (KR)

(73) Assignee: Electronics and Telecommunications Research Institute, Daejeon (KR)

(21) Appl. No.: 18/190,617

(22) Filed: Mar. 27, 2023

(30) Foreign Application Priority Data
Mar. 28, 2022 (KR) 10-2022-0037822
Sep. 13, 2022 (KR) 10-2022-0114890
Feb. 27, 2023 (KR) 10-2023-0025690

(51) Int. Cl. A61B 5/145; A61B 5/1455; A61B 5/01; A61B 5/00; A61B 5/107

(52) U.S. Cl. CPC A61B 5/14532; A61B 5/1455; A61B 5/01; A61B 5/6843; A61B 5/7246; A61B 5/1072; A61B 5/1075; A61B 5/1079; A61B 5/0095; A61B 5/6816; A61B 2562/0271; A61B 2562/0238; A61B 2562/0247

(57) ABSTRACT

Disclosed are a noninvasive blood glucose measurement apparatus and method using multiple sensors. The noninvasive blood glucose measurement apparatus includes: a first light source unit configured to irradiate light to the biological tissue, a second light source unit configured to irradiate light to the biological tissue, a first receiving unit configured to receive a photoacoustic and/or light transmission signal generated by the light, a second receiving unit configured to receive a light reflection signal reflected by the biological tissue, and a measurement unit configured to measure light reflection characteristics of the biological tissue by using the light reflection signal, measure photoacoustic and/or light transmission characteristics of the biological tissue, and measure blood glucose of the biological tissue on the basis of the light reflection characteristics and the photoacoustic and/or light transmission characteristics.

### CROSS-REFERENCE TO RELATED APPLICATIONS

This application claims priority to and the benefit of Korean Patent Applications, No. 10-2022-0037822 filed on Mar. 28, 2022, No. 10-2022-0114890 filed on Sep. 13, 2022, and No. 10-2023-0025690 filed on Feb. 27, 2023, the disclosures of which are incorporated herein by reference in their entirety.

### BACKGROUND

### 1. Field of the Invention

The present invention relates to a blood glucose measurement technology, and more particularly, to a blood glucose measurement apparatus and method capable of noninvasively measuring a blood-glucose concentration in biological tissue of the distal part such as an earlobe, a hand, or a foot of the human body, or any soft tissue.

### 2. Discussion of Related Art

Various techniques have been studied for non-drawing blood glucose measurements, which enable diabetic patients to avoid the pain of drawing blood. The non-drawing blood glucose measurement technique is largely classified into a minimally invasive method and a noninvasive method according to whether a glucose measurement sensor is inserted into biological tissue of the human body. The minimally invasive method uses a small probe or an implant-type sensor to measure a glucose concentration in interstitial fluid, which is body fluid between tissue cells outside the blood vessels, and thus can generally measure blood glucose without bleeding. The noninvasive measurement method is an innovative technique for measuring a glucose concentration outside the skin tissue without any tissue damage, and includes, for example, an in vitro body fluid-type noninvasive measurement technique for measuring the concentration of glucose contained in the body fluid discharged outside the body such as tears and sweat.

In order to measure a signal in biological tissue of the human body by the noninvasive measurement method, a physical signal needs to be applied thereto. For the applied physical signal, various types such as light, heat, electromagnetic, ultrasound, and fusion signals are used. A signal measurement sensor may be used in almost any part outside the human body, but is mainly being developed in a form that can be attached to or worn on a finger, an earlobe, an ear, a wrist, an arm, an abdomen, or the like. In particular, since the finger and the earlobe are considered as distal end tissues of the human body where the sensor is more conveniently worn and measured, many sensors currently on the market or being developed are being developed to have a form (for example, pivotal tongs) suitable for being worn on these biological tissues.

### SUMMARY OF THE INVENTION

The present invention is directed to providing a noninvasive blood glucose measurement apparatus and method using multiple sensors capable of noninvasively measuring a blood-glucose concentration in biological tissue such as an earlobe or a finger of the human body.

The present invention is also directed to providing a probe for noninvasive blood glucose measurement developed for effective use of the noninvasive blood glucose measurement apparatus and method using multiple sensors.

Noninvasive blood glucose measurement is generally based on the measurement of reflection and transmission characteristics of a physical signal in soft tissue containing blood glucose. The soft tissue in the human body refers to tissue other than bone or cartilage, such as skin, muscle, fat, fibrous tissue, lymphatic tissue, and blood vessels, and the composition of the soft tissue has a significant effect on noninvasive blood glucose measurement. A finger, a hand, and an earlobe have long been used as tissue parts (biological tissue) of the human body suitable for noninvasive blood glucose measurement, in particular, the earlobe is composed of rough muscle tissue and adipose tissue, is very flexible, and has a large amount of blood supply, and thus is one of the suitable parts for noninvasive blood glucose measurement.

The gist of the present invention is to measure blood glucose on the basis of light reflection characteristics and characteristics of at least one of photoacoustic transmission and light transmission, which are derived by multiple types of measurement methods using multiple sensors for relatively thin soft tissue such as an earlobe in body tissue (hereinafter, the phrase “at least one of photoacoustic transmission and light transmission” and similar phrases are used interchangeably with the phrase “photoacoustic and/or light transmission” and similar phrases).

To this end, a noninvasive blood glucose measurement apparatus and method according to the present invention include a multi-sensor probe configured to measure blood glucose noninvasively by coming into contact with both sides of biological tissue, for example, an earlobe. The multi-sensor probe may further include at least one of a means for irradiating light of a multi-wavelength channel to a biological tissue, a means for receiving backscattered/reflected light (hereinafter referred to as a “light reflection signal”) that is generated as the irradiated light passes through the biological tissue, a means for receiving light (hereinafter referred to as a “light transmission signal”) that is irradiated and has passed through the biological tissue and/or a means for receiving a photoacoustic transmission signal generated and transmitted in biological tissues by irradiated light, and a means for measuring a temperature and/or a thickness.

In particular, in the present invention, a blood glucose level in the body may be measured noninvasively by distinguishing a wavelength region for a light reflection signal and a wavelength region for a photoacoustic and/or light transmission signal on the basis of scattering and absorption characteristics of near-infrared light for biological tissue of the human body and light transmission and absorption characteristics according to a change in glucose concentration and selectively applying a wavelength (or wavelength channel) having a high reactivity to glucose and a reference wavelength (or reference wavelength channel) having a low reactivity to glucose in each wavelength region. In this case, a ratio of a signal of the wavelength having a high glucose reactivity to a signal of the reference wavelength in two wavelength regions may show a signal change rate in opposite directions according to an increase in a blood glucose level.

That is, in the present invention, a blood glucose level may be evaluated more accurately and noninvasively by measuring the blood glucose level in a dual manner on the basis of different noninvasive blood glucose measurement methods in two wavelength regions with respect to the same region of biological tissue (e.g., an earlobe), and additionally reflecting at least one of temperature and thickness measurement results for the biological tissue (hereinafter, the phrase “at least one of the temperature and the thickness” and similar phrases are used interchangeably with the phrase “the temperature and/or the thickness” and similar phrases).

The present invention uses a combination of multiple types of non-invasive measurement methods using multiple sensors, and provides a solution differentiated from the related art as follows.
1) Blood glucose is measured using “light reflection characteristics,” that is, the characteristics of light received by a detector after being multi-scattered, transmitted, and reflected except for a part of the light signal that is absorbed while an optical center path of the light irradiated to the biological tissue (hereinafter, “the light irradiated to the biological tissue” will be used interchangeably with “irradiation light,” “sent light signal,” “output light signal,” or similar terms) reciprocally transmits both sides of the biological tissue through different paths.
2) A blood glucose level is evaluated on the basis of “photoacoustic transmission characteristics” based on two wavelength channels, which react differently to light absorption characteristics for blood glucose when a light signal with a frequency for photoacoustic generation is absorbed in the biological tissue. Blood glucose may be measured using “light transmission characteristics” of light (hereinafter, referred to as a light transmission signal), which is received after being irradiated and passing through the biological tissue, together with or as an alternative to the photoacoustic transmission characteristics described above.
3) In order to configure a noninvasive blood glucose measurement apparatus according to a multi-type method using multiple sensors, the irradiation light is set as a combination of a plurality of wavelength channels (e.g., four wavelengths of λ1, λ2, λ3, and λ4) in a near-infrared wavelength region (about 900 to 1700 nm) in which a light scattering rate in biological tissue is smaller than that in a wavelength region of 900 nm or less. The light reflection characteristics and the photoacoustic and/or light transmission characteristics may be simultaneously measured by irradiation light obtained by optically modulating a light source of the same wavelength by a combination of the plurality of wavelength channels. In addition, the light reflection characteristics for the tissue may be measured by combining a wavelength channel (e.g., λ1) having low reactivity to a change in glucose concentration and a wavelength channel (e.g., λ2 or λ4) having a high reactivity to a change in glucose concentration. In addition, the photoacoustic and/or light transmission characteristics for the tissue may be measured by combining a wavelength channel (e.g., λ3) having low reactivity to a change in glucose concentration and a wavelength channel (e.g., λ2 or λ4) having a high reactivity to a change in glucose concentration.
4) The blood glucose level in the tissue is further evaluated on the basis of multi-sensor data including the measurement of a temperature and/or a thickness of the biological tissue, together with the measurement of the light reflection characteristics and the photoacoustic and/or light transmission characteristics.

According to an aspect of the present invention, there is provided a noninvasive blood glucose measurement apparatus including a first light source unit disposed in a probe and configured to irradiate light of at least one wavelength to a biological tissue, a second light source unit disposed in the probe and configured to irradiate light of at least one wavelength, which is different from that of the light of the first light source unit, to the biological tissue, a first receiving unit disposed in the probe and configured to receive a transmission signal generated as the light irradiated to the biological tissue passes through the biological tissue, a second receiving unit configured to receive a light reflection signal generated as the light irradiated to the biological tissue is reflected by the biological tissue, and a measurement unit configured to measure light reflection characteristics of the biological tissue by using the light reflection signal received by the second receiving unit, measure transmission characteristics of the biological tissue by using the transmission signal received by the first receiving unit, and measure blood glucose of the biological tissue on the basis of the light reflection characteristics and the transmission characteristics.

Here, each of the first light source unit and the second light source unit may irradiate the biological tissue with light of two or more wavelength channels in a near-infrared wavelength region of 900 to 1700 nm in which an extinction coefficient, which is the sum of an absorption coefficient and a scattering coefficient, is relatively small as compared to other wavelength regions.

In the noninvasive blood glucose measurement apparatus of the present invention, the transmission signal received by the first receiving unit may be a photoacoustic transmission signal, and the measurement unit may be configured to measure photoacoustic transmission characteristics of the biological tissue by using the photoacoustic transmission signal received by the first receiving unit. Alternatively, the transmission signal received by the first receiving unit may be a light transmission signal, and the measurement unit may be configured to measure light transmission characteristics of the biological tissue by using the light transmission signal received by the first receiving unit.

In the noninvasive blood glucose measurement apparatus of the present invention, the second light source unit may irradiate light of a first wavelength channel, and light of a second wavelength channel having relatively a higher reactivity to glucose than the first wavelength channel, and the first light source unit may irradiate light of a third wavelength channel, and light of a fourth wavelength channel having a relatively higher reactivity to glucose than the third wavelength channel. In this case, the measurement unit may measure the light reflection characteristics of the biological tissue by combining a light reflection signal for the first wavelength channel and a light reflection signal for the second wavelength channel or the fourth wavelength channel. In addition, the measurement unit may measure the transmission characteristics of the biological tissue by combining a transmission signal for the third wavelength channel and a transmission signal for the second wavelength channel or the fourth wavelength channel.

The noninvasive blood glucose measurement apparatus of the present invention may further include a first sensor unit configured to measure a temperature of the biological tissue, wherein the measurement unit may measure the blood glucose of the biological tissue by reflecting (or using) the temperature of the biological tissue. In addition, the noninvasive blood glucose measurement apparatus of the present invention may further include a temperature sensor configured to monitor a temperature of at least one of the first light source unit, the second light source unit, the first receiving unit, and the second receiving unit, and a temperature sensor configured to monitor a change in ambient air temperature of the probe.

In addition, the noninvasive blood glucose measurement apparatus of the present invention may further include a second sensor unit configured to measure a thickness of the biological tissue, wherein the measurement unit may measure the blood glucose of the biological tissue by reflecting (or using) the thickness of the biological tissue.

Here, the second sensor unit may include a light-sending element disposed at a position of a first part of the probe, which is spaced apart from the biological tissue, two or more light-receiving elements disposed at different positions of the first part of the probe, which are spaced apart from the biological tissue, and a reflecting element disposed at a position of a second part of the probe, which is spaced apart from the biological tissue, and the measurement unit may be configured to measure the thickness of the biological tissue by measuring an intensity difference or ratio of light signals generated as light irradiated from the light-sending element is reflected by the reflecting element and then received by the two or more light-receiving elements. In addition, the second sensor unit may include a pressure sensor configured to measure a pressure applied by the probe to the biological tissue and output the measured pressure as an electrical signal, and the measurement unit may be configured to estimate the thickness of the biological tissue by using the electrical signal output from the pressure sensor.

In the noninvasive blood glucose measurement apparatus of the present invention, the second light source unit may include a light source element configured to irradiate light of different inclination angles to the biological tissue, and the second receiving unit includes a receiving element configured to receive the light reflection signal at different inclination angles at a position corresponding to the light source element.

In the noninvasive blood glucose measurement apparatus of the present invention, the measurement unit may measure the light reflection characteristics and the transmission characteristics of the biological tissue by distinguishing a wavelength region for measuring the light reflection characteristics and a wavelength region for measuring the transmission characteristics, and selectively applying at least one wavelength channel having a relatively low reactivity to glucose and at least one wavelength channel having a relatively high reactivity to glucose in the distinguished wavelength regions.

The probe of the noninvasive blood glucose measurement apparatus of the present invention has a new fastening structure, that is, fixed-type balanced fastening structure, in which a force (pressure) is always uniformly applied to each part of biological tissue. The probe may include an upper frame, a lower frame, and a moving frame that moves between the upper frame and the lower frame, and may have a structure in which the first and second light sources and the first and second receiving parts are installed in the upper and/or lower frames, so that the signal may be transmitted and detected more stably to the biological tissue. Specifically, the probe may include an upper frame, a lower frame spaced apart from the upper frame and fixed to the upper frame, and a moving frame that is located between the upper frame and the lower frame, has a space separated from the lower frame, into which the biological tissue is inserted, and moves between the upper frame and the lower frame so that the space separated from the lower frame is changed, wherein the moving frame may be configured to apply a pressure to the biological tissue by an elastic element placed between the moving frame and the upper frame. Here, the moving frame may further include a separation space expansion mechanism configured to widen the space separated from the lower frame when a force is applied against the elastic element. In the probe having such a configuration, the first light source unit, the second light source unit, and the second receiving unit may be installed in the upper frame, and the first receiving unit may be installed in the lower frame. In addition, the noninvasive blood glucose measurement apparatus may further include a pressure sensor between the elastic element and the upper frame.

According to another aspect of the present invention, there is provided a noninvasive blood glucose measurement method including irradiating light of one or more different wavelengths to a biological tissue, receiving a light reflection signal generated as the light irradiated to the biological tissue is reflected by the biological tissue, receiving a transmission signal generated as the light irradiated to the biological tissue passes through the biological tissue, and measuring light reflection characteristics of the biological tissue by using the light reflection signal, measuring transmission characteristics of the biological tissue by using the received transmission signal, and measuring blood glucose of the biological tissue on the basis of the light reflection characteristics and the transmission characteristics, wherein the transmission signal includes at least one of a photoacoustic transmission signal and a light transmission signal, and the transmission characteristics includes at least one of photoacoustic transmission characteristics and light transmission characteristics.

Here, the noninvasive blood glucose measurement method may further include measuring at least one of a temperature and a thickness of the biological tissue.

In the noninvasive blood glucose measurement method of the present invention, the estimating of the blood glucose may include deriving an average value by repeating the light reflection characteristics measurement and the transmission characteristics measurement multiple times, and deriving a blood glucose value by comparing the average value with a reference comparison table obtained in advance through prior learning data.

In addition, the noninvasive blood glucose measurement method of the present invention may further include repeatedly performing the deriving of the blood glucose value to output accumulated blood glucose value data for a predetermined period of time. As described above, the features briefly summarized with respect to the present invention are merely exemplary aspects of the detailed description for the embodiments of the present invention to be described later, and do not limit the scope of the present invention.

### BRIEF DESCRIPTION OF THE DRAWINGS

The above and other objects, features, and advantages of the present invention will become more apparent to those of ordinary skill in the art by describing exemplary embodiments thereof in detail with reference to the accompanying drawings, in which:

FIG. 1 is a diagram for describing an earlobe, a probe, and a main measurement part;

FIG. 2 is a diagram illustrating a configuration of a noninvasive blood glucose measurement apparatus according to one embodiment of the present invention;

FIG. 3A is a diagram for describing setting of a wavelength section together with graphs of an absorption coefficient and a scattering coefficient of biological tissue and an absorbance of a glucose aqueous solution in a near-infrared wavelength region;

FIG. 3B illustrates an example of a combination of wavelengths of a multi-channel light source for noninvasive blood glucose measurement;

FIG. 3C illustrates a measurement result of one embodiment according to the noninvasive blood glucose measurement based on a multi-channel wavelength combination;

FIG. 4 is a diagram for describing a probe configuration for noninvasive blood glucose measurement based on a multi-channel wavelength combination, and for describing transmission of a light reflection signal and a photoacoustic transmission signal;

FIG. 5 is a diagram for describing a correlation between a light alignment angle according to a thickness of a sample and a sensor alignment distance according to the light alignment angle;

FIG. 6A is a diagram for describing an arrangement structure of multiple sensors at one end of the probe according to one embodiment;

FIG. 6B is a diagram for describing an arrangement structure of multiple sensors at another end of the probe according to one embodiment;

FIG. 7 is a diagram for describing one embodiment of a thickness measurement sensor.

FIG. 8 is a flowchart of a noninvasive blood glucose measurement method according to an embodiment of the present invention;

FIG. 9 is a configuration diagram of a device to which the noninvasive blood glucose measurement apparatus according to an embodiment of the present invention is applied;

FIG. 10 is a configuration diagram of a conventional pivotal tongs-type probe;

FIG. 11 is a configuration diagram of a probe for noninvasive blood glucose measurement according to an embodiment of the present invention;

FIG. 12 is a configuration diagram of a probe for noninvasive blood glucose measurement according to another embodiment of the present invention;

FIG. 13 is a configuration diagram of an upper frame of the probe of FIGS. 11 and 12 viewed from a different direction;

FIG. 14 is an exemplary diagram of light spectrum measurement for describing measurement examples of light transmission and light sending/receiving based on a balanced fastening structure probe according to the present invention;

FIG. 15 is an exemplary diagram of multi-channel light wavelength-based glucose concentration measurement according to the present invention;

FIG. 16 is an exemplary diagram of a result of measuring biological tissue samples by a noninvasive blood glucose measurement method using a wavelength channel assignment method according to the present invention;

FIG. 17 is an exemplary diagram of thickness measurement for describing measurement of a thickness of biological tissue inserted into the probe according to the present invention; and

FIG. 18 is a schematic flowchart of a short-term and continuous blood glucose measurement process on the basis of light and photoacoustic signals, temperature, and thickness monitoring information acquired from the probe according to the present invention.

### DETAILED DESCRIPTION OF EXEMPLARY EMBODIMENTS

Hereinafter, a noninvasive blood glucose measurement apparatus and method according to embodiments of the present invention will be described with reference to the drawings. In the embodiments to be described below, for convenience of description, among biological tissue parts such as an earlobe, a part between fingers, and a part between toes, an earlobe is specified and described as a target biological tissue for measuring a blood-glucose concentration.

Terms used in the following description are used to merely describe exemplary embodiments of the present invention, but are not intended to limit the present invention. In the present specification, the singular forms include the plural forms unless the context clearly dictates otherwise. Further, it is noted that terms “comprises” and/or “comprising” used herein do not exclude the presence or addition of one or more other components, steps, operations, and/or elements in addition to stated components, steps, operations, and/or elements. In addition, the terms “reflection” and “transmission” as used herein in the description of a light signals are not intended to exclude absorption, multi-scattering characteristics in biological tissue.

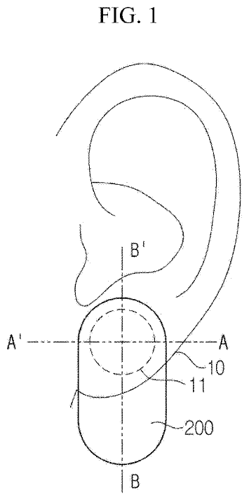

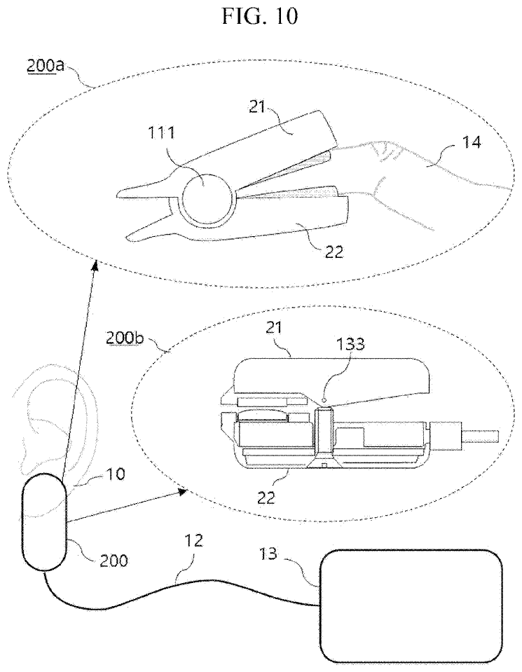

FIG. 1 is a diagram for describing an earlobe, a measurement region, and a probe, and illustrates a case in which a major axis of a probe 200 is mounted in parallel with a vertical (longitudinal) direction (B′-B) of an auricle. As shown in FIG. 1, the probe 200 worn on an earlobe 10 takes a certain part in the earlobe as a measurement region 11. The probe 200 coming in contact with both side surfaces of the earlobe having a certain thickness may generally have a shape of pivotal tongs in which a first part and a second part are elastically moved by a torsion spring or a compression spring. In the case of such a tongs structure, the probe 200 may have the major axis and a minor axis. In general, the probe may be mounted such that the major axis lies between a horizontal direction (A-A′) and the vertical direction (B-B′), and the noninvasive measurement region 11 may be smaller in size than the earlobe 10 regardless of the mounting shape of the probe. Later, in FIGS. 10 to 13, a creative balanced fastening-type probe that does not have such a pivotal tongs structure will be described.

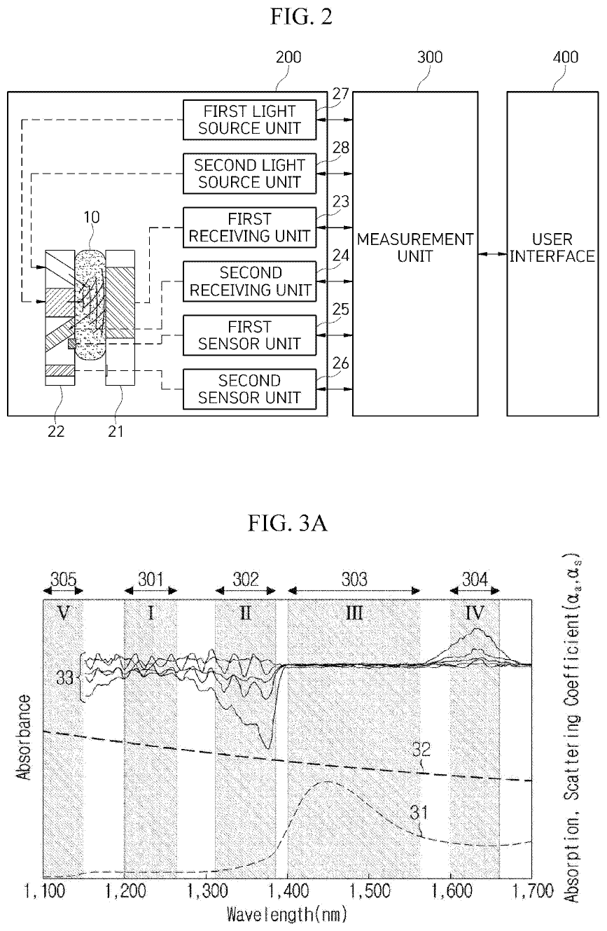

FIG. 2 is a diagram for describing a configuration of a noninvasive blood glucose measurement apparatus according to an embodiment of the present invention.

Referring to FIG. 2, the noninvasive blood glucose measurement apparatus according to the embodiment of the present invention includes the probe 200, a measurement unit 300, and a user interface 400. The probe 200 includes a first light source unit 27 configured to (vertically) irradiate light to a biological tissue 10 such as a part between fingers or an earlobe, a second light source unit 28 configured to irradiate light to the biological tissue 10 (at a predetermined angle), a first receiving unit 23 configured to receive a photoacoustic and/or light transmission signal, a second receiving unit 24 configured to receive a light reflection signal, a first sensor unit 25 configured to measure a surface temperature of the earlobe, and a second sensor unit 26 configured to measure a thickness of the earlobe. In addition, the probe 200 includes a first part 21 and a second part 22 physically coming into contact with tissue surfaces when worn on the earlobe 10, and the light source unit, the receiving unit, and the sensor units may be installed in the first part 21 and/or the second part 22 of the probe 200.

The first light source unit 27 may include light source elements of two or more different wavelength channels and an element for monitoring and/or controlling the output and/or temperature of the light sources, and irradiate light perpendicularly to the surface of the earlobe 10, but the present invention is not limited thereto.

The first light source unit 27 may irradiate light of a third wavelength channel having a relatively low reactivity to glucose and light of a fourth wavelength channel having a relatively high reactivity to glucose. Here, the low reactivity or high reactivity to glucose may mean relatively low or high reactivity when compared based on a certain value of reactivity to glucose or based on reactivity to glucose in a specific wavelength range, or may mean that a change in light absorbance is small below a predetermined value or the change is large above the predetermined value, and the criteria therefor may be determined by an individual or a business operator providing the corresponding technique.

The second light source unit 28 may include light source elements of two or more wavelength channels, and an element for monitoring and/or controlling the output and/or temperature of the light sources, and irradiate light to the surface of the earlobe 10 at a predetermined inclination angle, but the present invention is not limited thereto. The second light source unit 28 may include a plurality of light source elements capable of irradiating light to the earlobe at different inclination angles.

The second light source unit 28 may irradiate light of a first wavelength channel having a relatively low reactivity to glucose and light of a second wavelength channel having a relatively high reactivity to glucose. Here, the meaning of the low and high reactivities to glucose is similar to that described for the first light source unit 27 above.

Each of the first light source unit 27 and the second light source unit 28 may irradiate the earlobe with light of two or more wavelength channels in a near-infrared wavelength region of about 900 to 1700 nm in which an extinction coefficient that is the sum of an absorption coefficient and a scattering coefficient is the smallest.

In addition, the first receiving unit 23 includes an ultrasound sensor for receiving a photoacoustic transmission signal generated and transmitted as a light signal irradiated to the earlobe 10, which is a biological tissue, is absorbed in a part of the biological tissue. In addition or alternatively, the first receiving unit 23 may include a light-receiving sensor for receiving a light transmission signal generated as the light signal irradiated to the earlobe 10 is transmitted without being absorbed in the biological tissue.

The second receiving unit 24 may include a light-receiving element configured to receive a light reflection signal reflected while the light irradiated to the earlobe 10 passes through the earlobe 10. The light-receiving element may be aligned such that a surface (light-receiving surface) thereof has an inclination angle corresponding to a light-emitting angle of the second light source unit 28.

In addition, the first sensor unit 25 may include a temperature measuring element for measuring the temperature of the tissue by being in close contact with the skin of the earlobe 10.

The second sensor unit 26 may include a thickness measuring element for measuring a thickness of the biological tissue (i.e., the earlobe 10) between the first part 21 and the second part 22 of the probe.

In addition, the measurement unit 300 measures light reflection characteristics of the earlobe 10 using the light reflection signal received by the second receiving unit 24, and measures photoacoustic and/or light transmission characteristics of the earlobe 10 using the photoacoustic transmission signal (e.g., ultrasound signal) and/or the light transmission signal, which is received by the first receiving unit 23. Blood glucose of the earlobe 10 or a person concerned with the earlobe 10 is measured on the basis of the measured light reflection characteristics and photoacoustic and/or light transmission characteristics.

In this case, the measurement unit 300 may measure the light reflection characteristics and the photoacoustic and/or light transmission characteristics of the earlobe by distinguishing a wavelength region for measuring the light reflection characteristics and a wavelength region for measuring the photoacoustic and/or light transmission characteristics, and selectively applying at least one wavelength channel having a relatively low reactivity to glucose (reference wavelength channel) and at least one wavelength channel having a relatively high reactivity to glucose (blood glucose search wavelength channel) to the distinguished wavelength regions. According to an embodiment, the measurement unit 400 may measure the light reflection characteristics of the earlobe by combining a light reflection signal for the first wavelength channel and a light reflection signal for the second wavelength channel or the fourth wavelength channel, and may measure the photoacoustic and/or light transmission characteristics of the earlobe by combining a photoacoustic and/or light transmission signal for the third wavelength channel and a photoacoustic and/or light transmission signal for the second wavelength channel or the fourth wavelength channel.

Further, the measurement unit 300 may operate at least one of the light source units 27 and 28, the receiving units 23 and 24, and the sensor units 25 and 26 in the probe 200 according to a control timing, process the received signal, and store and output a measurement result. Furthermore, the measurement unit 300 may monitor an output intensity and a temperature of a multi-channel (multi-wavelength) light source included in the first and second light source units 27 and 28 and/or control a operation timing, a modulation frequency, an output intensity, a temperature, and the like.

According to an embodiment, the measurement unit 300 may sequentially process an ultrasound and/or light receiving signal of the first receiving unit 23 according to the control timing of the multi-channel light sources to derive and store a first result value for each wavelength channel. The measurement unit 300 may sequentially process a light reception signal of the second receiving unit 24 according to the control timing of the multi-channel light source to derive and store a second result value for each wavelength channel. The measurement unit 300 may sequentially process a temperature monitoring signal of the first sensor unit 25 according to the control timing of the multi-channel light source to derive and store a third result value for each wavelength channel. The measurement unit 300 may measure the thickness of the tissue between the first part 21 and the second part 22 of the probe 200 by operating the second sensor unit 26 at least once or more whenever a user wears the probe 200 on the earlobe 10 to derive and store a fourth result value. The measurement unit 300 may derive and store a first noninvasive blood glucose level for the blood glucose level in the tissue on the basis of a combination of the first to fourth result values derived from the first and second receiving units 23 and 24 and the first and second sensor units 25 and 26, and output the first noninvasive blood glucose level to the user interface 400.

The user interface 400 may include a display, a speaker, and a program for operating the same, which are integrated into a body of the noninvasive blood glucose measurement apparatus. Alternatively, the user interface 400 may include an external device such as a smartphone or a personal computer (PC) connected to the measurement unit 300 by a communication means, and a program application installed in the external device.

The user interface 400 may output the first noninvasive blood glucose level to the user, or may output a second blood glucose level by correcting the first noninvasive blood glucose level on the basis of personal data such as age, gender, and invasive blood glucose measurement data of the user. Furthermore, the user interface 400 may record blood glucose levels for each time and date, and compare the blood glucose levels with a currently measured blood glucose level to output a predicted blood glucose level.

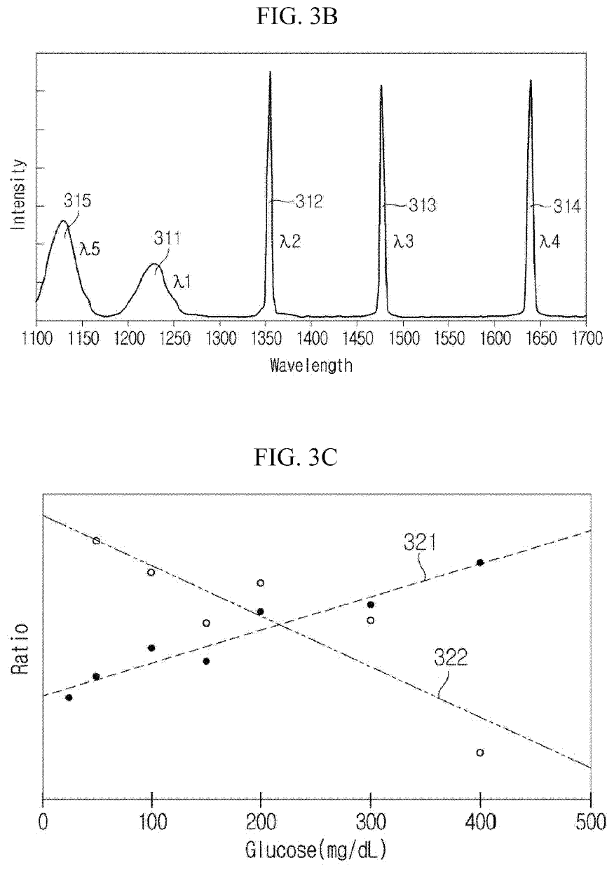

FIG. 3A is a diagram for describing setting of a wavelength section together with graphs of an absorption coefficient and a scattering coefficient of biological tissue and an absorbance of a glucose aqueous solution in a near-infrared wavelength region, shows characteristics of a light absorption coefficient 31 and a light scattering coefficient 32 for a general biological tissue of the human body in a partial section of a near-infrared wavelength region (about 1100 to 1700 nm) together with a light absorbance characteristic 33 according to a change in glucose concentration included in water, and illustrates a wavelength channel selection region according to the present invention.

In general, a light transmission in the biological tissue is attenuated due to scattering and absorption in various constituent materials constituting the tissue, and a transmittance is exponentially inversely proportional to the product of an extinction coefficient ae, which is the sum of an absorption coefficient aa and a scattering coefficient as, and a thickness t of the tissue. This may be expressed as Equation 1 below.

T˜exp(−ae*t); ae=aa+as [Equation 1]

In general, when light is transmitted in biological tissue, the extinction coefficient ae has a relatively small value in a near-infrared band having a wavelength in the range of 1100 to 1800 nm except for about 1490 nm, and thus the most excellent transmission characteristic may be expected along an optical path. Since the scattering coefficient as is much larger than the absorption coefficient aa in a region from about 480 to 1100 or about 1380 nm, optical characteristics in which light is not absorbed but scattered multiple times in the tissue are more easily used. A light signal that is multi-scattered in the biological tissue returns to an incident direction through a process of being repeatedly scattered backward or forward. Backscattering characteristics of a portion of the light signal may be measured by a reflection signal, and forward scattering characteristics of the incident light may be measured by a transmission signal.

Meanwhile, the generation of the photoacoustic signal for the biological tissue is based on the light absorption in the tissue component, and thus it is more advantageous for acoustic signal conversion in a local region with low light energy in the wavelength region having a relatively high absorption coefficient aa. As can be seen from the absorption coefficient graph 31 shown in FIG. 3A, it can be seen that, in the noninvasive photoacoustic measurement for the biological tissue, the absorption coefficient is larger in the near-infrared region of about 1150 nm or more than that of visible light and near-infrared light having wavelengths shorter than that thereof, and is further larger in the wavelength region of about 1400 nm or more.

As can be seen from the graph 33 shown in FIG. 3A of measuring the light absorbance according to a change in glucose concentration included in the water, it shows spectrum characteristics including an interference effect due to partial light reflection at one end of a cuvette, and a significant change is not shown in wavelength sections I and III according to an increase in glucose concentration, but characteristics in which absorbance is reduced in wavelength sections II and V and increased in a wavelength section IV appear.

In the embodiment of the present invention, a wavelength region of 4 channels or more is used from a region of 900 to 1700 nm in which the extinction coefficient, which is the sum of the absorption coefficient 31 and the scattering coefficient 32, is the smallest, for the transmission of the visible light and infrared light spectrum to biological tissue. In particular, as shown in FIG. 3A, light sources having wavelengths selected from wavelength sections I (301) and III (303) having a low reactivity to a glucose aqueous solution may be used as first and second reference signals, respectively, and light sources having wavelengths selected from wavelength sections II (302), IV (304), and V (305) having a relatively high reactivity to glucose may be used as first, second, and third blood glucose search signals, respectively.

When the characteristics of each wavelength section are described in more detail, the wavelength section I (301) has characteristics in which the light absorption coefficient is small, but the light scattering coefficient is relatively large, and a change in light absorbance according to a change in glucose concentration is relatively small, for the biological tissue of the human body. The wavelength section II (302) is a section having a relatively small extinction coefficient (the sum of the absorption coefficient and the scattering coefficient) compared to the wavelength section I (301) and has characteristics in which light absorbance is slightly and significantly decreased as the glucose concentration increases. The wavelength section III (303) is a section in which the extinction coefficient increases relatively greatly due to the increase in the absorption coefficient by moisture in the biological tissue of the human body, and has characteristics in which there is no change in light absorbance due to a change in glucose concentration. The wavelength section IV (304) is a section in which, as compared with the wavelength section II, the absorption coefficient is large and the scattering coefficient is small, but the extinction coefficient is similar, and has characteristics in which the light absorbance increases relatively greatly with the increase in glucose concentration. The wavelength section V (305) is a section in which, as compared with the other sections, the absorption coefficient is the smallest but the scattering coefficient is the largest, and has characteristics in which the light absorbance slightly increases with the increase in glucose concentration.

FIG. 3B is a diagram illustrating an example of a combination of wavelengths of the multi-channel light source for noninvasive blood glucose measurement, and as shown in FIG. 3B, center wavelengths of wavelength channels λ1 (311), λ2 (312), λ3 (313), λ4 (314), and λ5 (315) are located in the wavelength section I (301) of about 1200 to 1270 nm, the wavelength section II (302) of about 1310 to 1385 nm, the wavelength section III (303) of about 1400 to 1560 nm, the wavelength section IV (304) of about 1600 to 1660 nm, and the wavelength section V (305) of about 900 to 1150 nm, respectively.

For the light source for each wavelength channel, a relatively inexpensive diode light source, such as a light-emitting diode (LED), a laser diode (LD), a super luminescent diode (SLD), or the like, may be selectively used. In addition, each light source may have a different spectral half-width. As an example, as shown in FIG. 3B, a light source with a wide spectral half-width may be used for a first reference wavelength channel λ1 (311), and a light source with a narrow spectral half-width may be used for a second reference wavelength channel λ3 (313). In addition, a light source with a narrower spectral half-width may be used for the wavelength channels λ2 (311) and λ4(314) of first and second blood glucose search signals, and a light source with a wider spectral half-width may be used for a third blood glucose search wavelength channel λ5 (315). In addition, the center wavelengths of λ1(311), λ2(312), λ3(313), λ4(314), and λ5(315) may be differently selected in the ranges of the wavelength sections I, II, III, IV, and V, respectively, and widths of the spectral half widths of the light sources may also be differently selected.

Referring to FIGS. 3A and 3B, in the embodiment of the present invention, λ1 (301 and 311) and λ3 (303 and 313) may be selectively used as a reference wavelength in the light reflection measurement for the biological tissue of the earlobe, and λ2 (302 and 312), λ4 (304 and 314), and λ5 (305 and 315) may be selectively used as a wavelength for blood glucose search. Similarly, λ1 (301 and 311) and λ3 (303 and 313) may also be selectively used as the reference wavelength in the photoacoustic transmission measurement for the biological tissue of the earlobe, and λ2 (302 and 312), λ4 (304 and 314), and λ5 (305 and 315) may be selectively used as the wavelength for blood glucose search. In optical and photoacoustic methods, the reference wavelength channels are used to measure general characteristics of tissues, which are less sensitive or irrelevant to a blood-glucose concentration, and the blood glucose search channels are used to measure tissue characteristics including blood glucose information in a sample.

As described above, the apparatus according to the embodiment of the present invention is capable of measuring the blood glucose level while excluding the general characteristics of the biological tissue as much as possible by comparing and analyzing the measurement signals in the reference wavelength channels and the blood glucose search wavelength channels. Among various methods of comparing and analyzing measurement signals between the reference wavelength channels and the blood glucose search wavelength channels, as a simple example, the blood glucose level may be primarily derived from a difference or ratio of the two signals.

FIG. 3C is a diagram illustrating a measurement result of one embodiment according to the noninvasive blood glucose measurement based on a multi-channel wavelength combination, and illustrating an example of the result of measuring the change in glucose concentration (25 to 400 mg/dL) in a cuvette sample by each of the light transmission method and the photoacoustic method.

As shown in FIG. 3C, a trend line 321 obtained from ratio (λ5/λ1) data between the signal of the first reference wavelength λ1 and the signal of the third blood glucose search wavelength λ5 measured by the light transmission method may be expressed by a proportional equation between the measured noninvasive glucose value Y1 and the actual glucose concentration X as shown in Equation 2 below.

Y1=aX+b(a>0) [Equation 2]

In addition, a trend line 322, which is obtained from ratio (λ4/λ3) data between the signal of the second reference wavelength λ3 and the signal of the second blood glucose search wavelength λ4 measured by the photoacoustic method, may be expressed by a proportional equation between another noninvasive glucose measurement value Y2 and an actual glucose concentration X as shown in Equation 3 below.

Y2=cX+d(c<0) [Equation 3]

The data shown in FIG. 3C shows the result that does not reflect the correction according to the temperature and/or thickness of the sample, but it can be seen that blood glucose levels may be measured and compared multiple times by the noninvasive light transmission and photoacoustic measurement method according to the present invention.

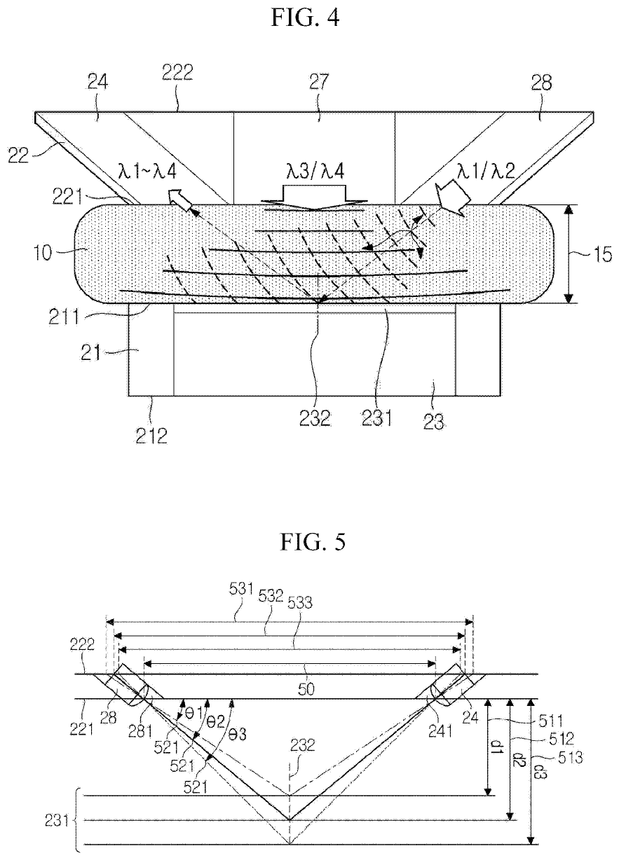

FIG. 4 is a diagram for describing a probe configuration for noninvasive blood glucose measurement based on a multi-channel wavelength combination, and a light reflection signal, and a photoacoustic and/or light transmission signal transferring technique, and illustrates a cross section of the probe 200 in the direction of the axis (A-A′) in FIG. 1, and the probe 200 is configured such that the earlobe 10 is in close contact between the first part 21 and the second part 22 of the probe 200.

As shown in FIG. 4, the first part 21 of the probe 200 is configured to include the first receiving unit 23 described in FIG. 2, and the second part 22 of the probe 200 is configured to include the first light source unit 27, the second light source unit 28, and the second receiving unit 24 described in FIG. 2.

A light module for the first light source unit 27 may be composed of two LDs or LEDs emitting the second reference wavelength λ3 and the second blood glucose search wavelength λ4. Here, a collimator lens for condensing light or making parallel light by adjusting a divergence angle of emitted light may also be assembled to the first light source unit 27.

A light module for the second light source unit 28 may be composed of two LDs or LEDs emitting the first reference wavelength λ1 and the first blood glucose search wavelength λ2. Here, a collimator lens for condensing light or making parallel light by adjusting a divergence angle of emitted light may be assembled to the second light source unit 28.

As another example, the third blood glucose search wavelength λ5 may be used in place of the first blood glucose search wavelength λ2 or the second blood glucose search wavelength λ4. In addition, a thermistor for monitoring a temperature of an optical element mount may be assembled together on a thermoelectric cooling element (TEC) or a heater element for temperature control in the optical element in the light module.

The first receiving unit 23 may include an ultrasound sensor and/or a light transmission signal receiving photodiode, and the second receiving unit 24 may include a single photodiode capable of receiving all light signals of near-infrared wavelength channels λ1 to λ5, that is, light reflection signals transmitted through the earlobe 10 and reflected. In the description of FIG. 4 below, the first reception unit 23 will be described from the viewpoint of photoacoustic transmission for simplicity of description, but the description may be applied to the case of light transmission.

As shown in FIG. 4, a size (a diameter or a width) of the light module of the first light source unit 27 is smaller than that of the ultrasound sensor of the first receiving unit 23, and an output light's central axis is disposed to be aligned with a center portion of the ultrasound sensor so that a traveling direction of the light is perpendicular to the surface 221 of the probe that comes into contact with the skin of the earlobe. The second light source unit 28 and the second receiving unit 24 are arranged on both sides of the first light source unit 27 to have a predetermined inclination, and each of the inclined surfaces may be disposed to have an angle so that a direction in which the center of a light transmission signal output from the second light source unit 28 travels and a direction in which the center of a light reception signal incident to the second receiving unit 24, that is, the light reflection signal travels are directed to the first receiving unit 23, that is, a center portion 232 of an impedance matching layer 231 assembled on the incident surface of the ultrasound sensor. The impedance matching layer 231 of the ultrasound sensor may be made of a material that has an impedance that can minimize the reflection of ultrasound energy between the earlobe biological tissue and the ultrasound receiving element, and at the same time has a uniform surface that reflects light of wavelengths λ1 and λ2 relatively greatly.

As earlier described in FIG. 2, the light sources for each wavelength channel in the first and second light source units 27 and 28 may be controlled to output a modulated wave or a continuous pulse wave at a specific frequency for a certain period of time by the measurement unit 300 and may be controlled to sequentially output a plurality of wavelength channels at a predetermined time interval. Some of the light signals of the wavelengths λ3 and λ4 output from the first light source unit 27 are absorbed at a shallow depth of the earlobe 10 below the skin contact surface 221 of the second part 22 of the probe due to a relatively high absorption coefficient to generate a photoacoustic transmission signal, that is, an ultrasound signal, and the generated ultrasound signal passes through the earlobe 10 and is received by the first receiving unit 23 located on the first part 21 of the probe having a contact surface 211 on the opposite skin of the earlobe. In addition, a small amount of energy of some of the light signals of the wavelengths λ3 and λ4, which are scattered and reflected in the tissue of the earlobe 10, may be received by the second receiving unit 24. Since the light signals of the wavelengths λ1 and λ2 output from the second light source unit 28 have a relatively low absorption coefficient and a high scattering characteristic, a portion of the scattered and transmitted light energy is transmitted to a depth of several mm under the skin of the earlobe without significant loss, and reflected from a wide surface of the impedance matching layer 231 of the ultrasound sensor 23 toward the opposite surface of the earlobe (i.e., 221), so that a small amount of partial light energy may be received by the second receiving unit 24. In addition, some energy of the light signals of the wavelengths λ1 and λ2 output from the second light source unit 28 is absorbed in the tissue of the earlobe 10 to generate a photoacoustic transmission signal, and the photoacoustic transmission signal may pass through the earlobe 10 and be received by the first receiving unit 23.

FIG. 5 is a diagram for describing a correlation between a light alignment angle according to a thickness of the biological tissue (i.e., the earlobe 10) and a sensor alignment distance according to the light alignment angle, and is a diagram for describing a correlation between the inclination angle of each of the second light source unit 28 and the second receiving unit 24 and a distance 15 between both parts 21 and 22 of the probe 200, that is, the thickness of the earlobe 10 in FIG. 4.

FIG. 5 shows the relationship between thicknesses 511 to 513 and inclination angles 521 to 523 for symmetrically matching a traveling direction of a light transmission signal output from the second light source unit 28 and a traveling direction of a light reception signal incident to the second receiving unit 24 with respect to the center portion 232 of the impedance matching layer 231 of the ultrasound sensor 23. That is, inclination angles corresponding to thicknesses d1 (511), d2 (512), and d3 (513) are equal to θ1 (521), θ2 (522), and θ3 (523), respectively.

Unlike a conventional measurement method in which only transmission characteristics due to light scattering in the earlobe tissue are mainly received, in the present invention, light scattered in the tissue is additionally reflected from the surface (i.e., 231) of the ultrasound sensor 23 and is aligned so that the light can be absorbed and scattered again while passing through the tissue in the direction of the second receiving unit 24. The light scattering is largely divided into Rayleigh scattering due to particles smaller than the wavelength and Mie scattering due to particles larger than the wavelength. Generally the biological tissue is composed of cell tissues larger than the near-infrared wavelength, and thus the light scattering in the present invention occurs mainly according to a Mie scattering effect. The Mie scattering shows a characteristic in that energy scattered forward increases as a diameter of the particle relative to the wavelength increases. Accordingly, since a large portion of the energy multi-scattered in the biological tissue of the earlobe, which is several mm thick, is still distributed near the center of a light incident path, matching the inclination angles of the light transmission and reception is more advantageous for receiving light transmission energy sufficiently. To this end, as shown in FIG. 4, the second light source unit 28 and the second receiving unit 24 located at both regions of the first light source unit 27 may be configured as a pair of light-sending element and light-receiving element having different inclination angles.

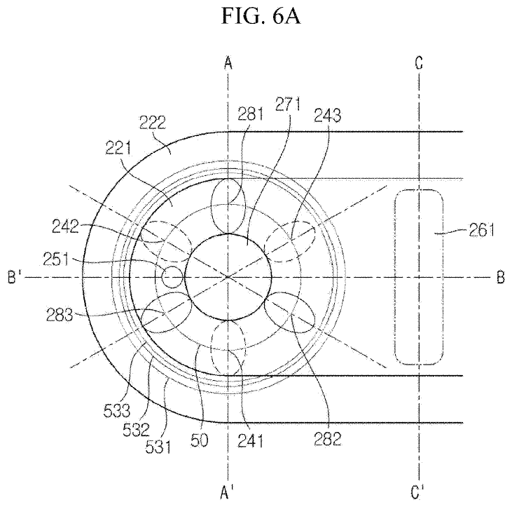

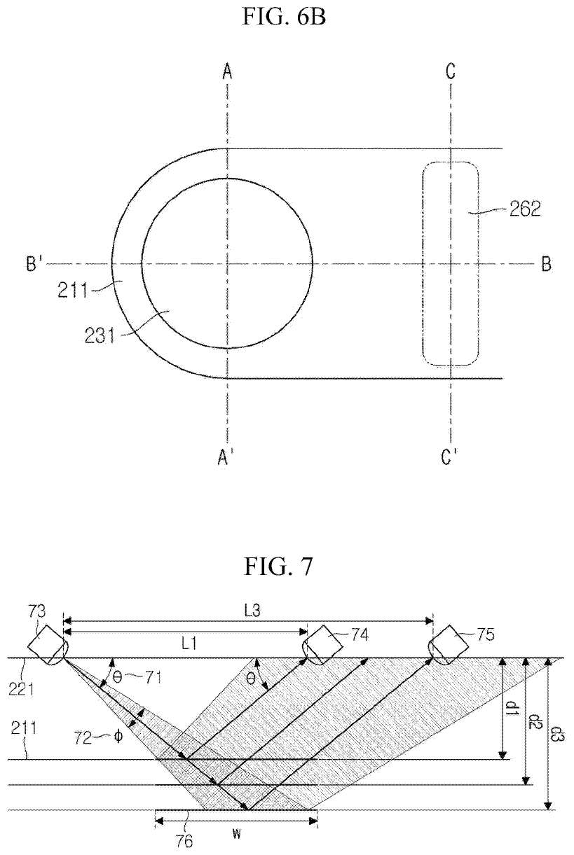

FIG. 6A is a diagram for describing an arrangement structure of multiple sensors in the second part of the probe according to one embodiment, and FIG. 6B is a diagram for describing an arrangement structure of multiple sensors in the first part of the probe according to one embodiment. This is a diagram showing an example of the probe in which the second light source unit 28 and the second receiving unit 24 are composed of three pairs of light-transmitting and receiving elements having different inclination angles, as described in FIG. 5.

FIG. 6A mainly shows a region in which each element may be installed when the second part 22 of the probe is viewed from a direction of a surface of the second part 22 in contact with the earlobe 10, and a portion of the second part 22 of the probe coming into contact with the earlobe 10 is illustrated in directions of the axes A-A′ and B-B′ indicated in FIG. 1. As shown in FIG. 6A, the surfaces 221 and 222 correspond to the surface 221 of the second part 22 of the probe in contact with the tissue of the earlobe 10 and the other surface 222 thereof, respectively, in FIG. 4. Three regions 281, 282, and 283 for installing the second light source unit 28 and three regions 241, 242, and 243 for installing the second receiving unit 24 are arranged to face each other around an installation region 271 of the first light source unit 27. As shown in FIG. 5, three pairs of light sending and receiving elements for the second light source unit 28 and the second receiving unit 24 are configured to face each other around the installation region 271 of the first light source unit 27 to have three different inclination angles according to inclination angles θ1 to θ3 centered on three earlobe thicknesses d1 to d3. As shown in FIG. 5, the illustrated elliptical shape represents the shape of the regions 241 and 281 in a structure installed at an inclination angle, and all of ellipses 241 to 243 and 281 to 283 may be arranged so that centers thereof lie on a circle 50. Here, a diameter of the circle 50 may be configured to be about 10 mm or less, which is a desired maximum expected size of the measurement region 11 in the earlobe 10 as described in FIG. 1. In addition, in FIG. 6A, a diameter of each of the other circles 531 to 533 indicates an interval between both ends when the second light source unit 28 and the second receiving unit 24 are assembled to have three inclination angles on the surface 222 of the second part 22 of the probe, as shown in FIG. 5. An element 251 represents an installation region of a thermistor for measuring the surface temperature of the earlobe.

FIG. 6B illustrates the contact surface 211 (see FIG. 4) and an installation position 231 of the ultrasound sensor for the first receiving unit 23 looking at the first part 21 of the probe from the direction of the contact surface of the earlobe 10.

In FIGS. 6A and 6B, rectangular shapes 261 and 262 long in a direction of an axis (C-C′) indicate an installation region of a thickness measurement sensor, that is, the second sensor unit 26. The thickness measurement sensor will be described in detail with reference to FIG. 7.

FIG. 7 is a diagram for describing one embodiment of the thickness measurement sensor.

The thickness measurement sensor illustrated in the embodiment of FIG. 7 includes a light-sending element 73 and two light-receiving elements 74 and 75 installed on the second part 22 of the probe by avoiding the earlobe 10 between both parts 21 and 22 of the probe, and a reflecting element, e.g., a reflection mirror 76, installed on the first part 21 of the probe. The light-sending element 73 is installed to have a specific inclination angle θ(71) with respect to the surface 221 of the second part 22 of the probe, and the two light-receiving elements 74 and 75 are installed at distances L1 and L3 from the light-sending element 73, respectively, to have the same inclination angle and an angle opposite to the angle θ. In addition, the reflection mirror 76 formed on the surface 211 of the first part 21 of the probe is designed to have a width w (76) sufficient to cover light reflection with respect to the thickness changes d1 to d3 in the direction of the axis (C-C′).

Light emitted from the light-sending element 73 has a radiation angle φ(72) of a predetermined size and is irradiated toward the reflection mirror 76. It is configured such that an intensity of a signal received by the receiving element 74 has the greatest value when a distance between the surface 211 of the first part of the probe and the surface 221 of the second part is d1, intensities of signals received by the receiving elements 74 and 75 have the same value when the distance is d2, and an intensity of a signal received by the receiving element 75 has the greatest value when the distance is d3.

Here, d1 and d3 mean the minimum thickness and the maximum thickness of the earlobe 10 to be measured, and the output radiation angle φ (72) of the light-sending element 73 may be set so that signals received by the light-receiving elements 74 and 75 have the same intensity at d2 corresponding to the intermediate value between d1 and d3.

The light-sending element 73 may output and irradiate a continuous wave or pulse wave having a certain intensity, and the measurement unit 300 (see FIG. 2) may measure a difference or ratio of intensities of the signals received by the two light-receiving elements 74 and 75 and measure or evaluate an actual thickness when being attached on the earlobe according to the thicknesses d1 to d3 on the basis of pre-trained look-up data.

Although it has been described above with reference to FIGS. 1 to 7 that blood glucose of biological tissue is measured using the light reflection characteristics and the photoacoustic and/or light transmission characteristics, the noninvasive blood glucose measurement apparatus of the present invention may also measure the blood glucose of the biological tissue using only the light reflection characteristics. For example, a noninvasive blood glucose measurement apparatus according to another embodiment of the present invention may measure blood glucose of biological tissue using only light reflection characteristics by removing the first receiving unit 23 shown in FIGS. 2 and 4. That is, the noninvasive blood glucose measurement apparatus according to another embodiment of the present invention may include a first light source unit 27 configured to irradiate light of a third wavelength channel and a fourth wavelength channel to biological tissue, a second light source unit 28 configured to irradiate light of a first wavelength channel and a second wavelength channel to the biological tissue at a predetermined inclination angle, a second receiving unit 24 configured to receive a light reflection signal in which the light irradiated to the biological tissue is reflected while passing through the biological tissue, and a measurement unit 300 configured to divide the first to fourth wavelength channels into at least one reference wavelength channel having a relatively low reactivity to glucose and at least one blood glucose search wavelength channel having a relatively high reactivity to glucose, measure light reflection characteristics of the biological tissue using the light reflection signals in the reference wavelength channel and the blood glucose search wavelength channel, and measure the blood glucose of the biological tissue on the basis of the light reflection characteristics. Here, in order to effectively secure a reception strength of a light reflection signal, the noninvasive blood glucose measurement apparatus according to another embodiment of the present invention may be implemented to reflect light signals of wavelength channels λ1 to λ4, which have passed through the biological tissue, toward the second receiving unit 24 by including a light reflecting element at a position of the first receiving unit 23. That is, the reception strength of the light reflection signal received by the second receiving unit 24 may be increased by removing the first receiving unit 23 and providing the reflecting element at the corresponding position and reflecting the light of the first to fourth wavelength channels passing through the biological tissue toward the second receiving unit 24. Of course, the noninvasive blood glucose measurement apparatus according to another embodiment of the present invention may include all the contents described in FIGS. 1 to 7.

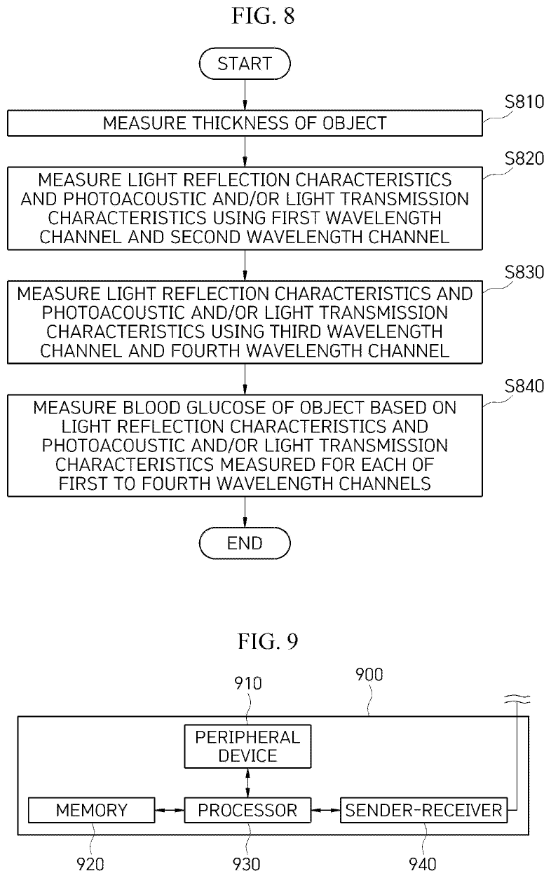

FIG. 8 is a diagram illustrating a noninvasive blood glucose measurement method according to an embodiment of the present invention as a flowchart, and is a diagram illustrating a process of measuring blood glucose in the apparatus described with reference to FIGS. 1 to 7 as a flowchart.

Referring to FIG. 8, in the noninvasive blood glucose measurement method according to the embodiment of the present invention, a measurement unit 300 recognizes a measurement start command of a user or a preset periodic repetitive measurement command in a state in which the probe 200 is worn on an earlobe 10, and operates a second sensor unit 26 to measure a thickness of the earlobe in contact with both parts of the probe and derive a first result value (S810).

Thereafter, light reflection characteristics and photoacoustic and/or light transmission characteristics are measured using a first wavelength channel and a second wavelength channel (S820). That is, by operating the second light source unit 28 to irradiate the first wavelength channel and the second wavelength channel to the earlobe 10, and receiving a photoacoustic and/or light transmission signal through the first receiving unit 23 and receiving a light reflection signal through the second receiving unit 24, light reflection characteristics and photoacoustic and/or light transmission characteristics for the first wavelength channel and the second wavelength channel are measured, and accordingly, a second result value is derived. In this case, in operation S820, a temperature of the earlobe, which is biological tissue, may also be measured.

Thereafter, light reflection characteristics and photoacoustic and/or light transmission characteristics are measured using a third wavelength channel and a fourth wavelength channel (S830). That is, by operating the first light source unit 27 to irradiate the third wavelength channel and the fourth wavelength channel to the earlobe, and receiving a photoacoustic signal and/or a light signal through the first receiving unit 23 and receiving a light reflection signal through the second receiving unit 24, light reflection characteristics and photoacoustic and/or light transmission characteristics for the third wavelength channel and the fourth wavelength channel are measured, and accordingly, a third result value is derived. In this case, in operation S830, a temperature of the earlobe may also be measured.

When the light reflection characteristics and the photoacoustic and/or light transmission characteristics for each of the first to fourth wavelength channels are measured through the above-described process, blood glucose of the earlobe is measured on the basis of the light reflection characteristics and the photoacoustic and/or light transmission characteristics measured by each of the first to fourth wavelength channels (S840). Here, it is possible to measure the blood glucose of the earlobe by reflecting the thickness and temperature measured for the earlobe. Furthermore, customized temperature and thickness correction may be automatically performed for each person to be measured.

The operation S840 is further described. A learning matrix is created and trained based on multi-data including the first to third result values derived through operations S810 to S830 described above and data log values stored in the noninvasive blood glucose measurement apparatus or terminal. That is, multi-data-based signal processing is performed, a final blood glucose level of the earlobe is evaluated on the basis of the signal processing, the evaluated blood glucose level of the biological tissue is accumulated in an existing data log, a new evaluation result is stored, and the new evaluation result is output to the corresponding user.

The measurement unit 300 may include and store user's invasive blood glucose measurement values for calibrating the noninvasive blood glucose measurement apparatus in the existing data log, and may also output new measurement data including existing data according to the requirements of the user interface 400.

Although a description is omitted in the method of FIG. 8, the noninvasive blood glucose measurement method according to an embodiment of the present invention may include all the contents described with reference to FIGS. 1 to 7.

The noninvasive blood glucose measurement apparatus and method according to the embodiment described above may provide a noninvasive blood glucose measurement technique based on a multiple sensor-based technique, which is completely different from a conventional earlobe-type noninvasive blood glucose sensor, more accurately measure a blood-glucose concentration of biological tissue in a noninvasive manner by mutually combining light reflection characteristics together with photoacoustic and/or light measurement for biological tissue such as an earlobe, and the measured blood-glucose concentration may be corrected by reflecting the thickness and temperature of the biological tissue in the blood glucose measurement history as necessary.

FIG. 9 is a diagram for describing a device 900 in which the noninvasive blood glucose measurement apparatus and method according to the embodiment of the present invention is implemented. The device 900 may include a memory 920, a processor 930, a sender-receiver 940, and a peripheral device 910, but may additionally include other components. The device 900 may be, for example, a fixed network management device (e.g., a server, a PC, or the like).

More specifically, the device 900 of FIG. 9 may be an exemplary hardware/software architecture such as a blood glucose measurement apparatus, a blood glucose management apparatus, a blood glucose measurement and prediction terminal, a user-customized blood glucose management apparatus, and the like. As an example, the memory 920 may be a non-movable memory or a movable memory. In addition, as an example, the peripheral device 910 may include a display, a global positioning system (GPS), or other peripheral devices.

As an example, The device 900 may include a communication circuit such as the sender-receiver 940 and perform communication with an external device based on this.

As an example, the processor 930 may be at least one of a general processor, a digital signal processor (DSP), a DSP core, a controller, a microcontroller, application specific integrated circuits (ASICs), field programmable gate array (FPGA) circuits, any other types of integrated circuits (ICs), and one or more processors related to state machine. That is, the processor may be a hardware/software component for controlling the above-described device 900. In addition, the processor 930 may modularize and perform functions of the measurement unit 300 and the user interface 400 of FIG. 2 described above.

The processor 930 may execute computer-executable instructions stored in the memory 920 in order to perform various essential functions of the noninvasive blood glucose measurement apparatus. As an example, the processor 930 may control at least one of signal coding, data processing, power control, input/output processing, and communication operation. In addition, the processor 930 may control a physical layer, a media access control (MAC) layer, and an application layer. In addition, as an example, the processor 930 may perform authentication and security procedures in an access layer and/or an application layer.

As an example, the processor 930 may perform communication with other apparatuses through the sender-receiver 940. As an example, the processor 930 may control the noninvasive blood glucose measurement apparatus to perform communication with other apparatuses via a network by executing the computer-executable instructions. That is, communication performed in the present invention may be controlled. As an example, the sender-receiver 940 may transmit a radio frequency (RF) signal through the antenna and transmit signals based on various communication networks. As an example, multiple input and multiple output (MIMO) technology, beamforming, and the like may be applied as antenna technology, and the present invention is not limited to the above-described embodiments. In addition, the signal transmitted/received through the sender-receiver 940 may be modulated and demodulated, and controlled by the processor 930.

Although exemplary methods of the present invention are represented in a series of steps for clarity of a description, the exemplary methods are not intended to limit the sequence of steps. Some steps may be performed simultaneously or may be performed in a different order as necessary. In order to implement the method presented by the present invention, an additional step may be added to the exemplary method, some steps may be omitted, or some steps are omitted and an additional step may be added to the exemplary method.

Various embodiments of the present invention are not listed in all possible combinations, but are for describing representative aspects of the present invention, and matters described in the various embodiments may be applied independently or may be applied as a combination of two or more.

In addition, various embodiments of the present invention may be implemented by hardware, firmware, software, or a combination thereof. In the case of implementing the present invention by hardware, the present invention may be implemented with one or more ASICs (Application Specific Integrated Circuits), DSPs (Digital Signal Processors), DSPDs (Digital Signal Processing Devices), PLDs (Programmable Logic Devices), FPGAs (Field Programmable Gate Arrays), general processors, controllers, microcontrollers, microprocessors, or the like.

The scope of the present invention includes software or machine-executable instructions (e.g., operating systems, applications, firmware, programs, or the like) that allow operations according to methods of various embodiments to be executed on a device or a computer, and a non-transitory computer-readable medium which stores such software and instructions and is executable on a device or a computer.

Now, an actual implementation form of the probe 200 (see FIG. 2), which is one of the components of the above-described noninvasive blood glucose measurement apparatus and method using multiple sensors according to an embodiment of the present invention, will be described.

As mentioned above, basically, the probe may be implemented in a pivotal tongs structure as shown in FIG. 10. In general, a diagnostic apparatus for measuring bio-signals noninvasively includes a probe 200 worn on the human body, a data communication element 12, and a bio-recorder 13. The probe 200 is a device worn on the part of the human body to be measured to acquire a bio-signal, and the data communication element 12 indicates a wired cable or a wireless communication means for transmitting data measured by the probe to the bio-recorder 13. The bio-recorder 13 may include all of the functions of controlling the probe, processing a measurement signal, and displaying a measurement result, or may include only some of these functions.

Light source units (27 and 28 of FIG. 2), receiving units (23 and 24 of FIG. 2), and sensor units (25 and 26 of FIG. 2) are installed in a first part 21 and a second part 22 of a probe 200a, which has a pivotal tongs structure, shown in the upper part of FIG. 10. The pivotal tongs-type probe 200a having such a structure is implemented in a scissors shape rotating around an intersection part 111 of the first part 21 and the second part 22, and thus there is a limitation in fixing each part of the biological tissue, such as a finger 14 located between the first part 21 and the second part 22, with a uniform force (pressure). A structure of both parts of the probe may be designed to apply a uniform force according to a specific thickness of the biological tissue, but when biological tissue of a smaller or larger thickness is inserted between the both parts, the force applied to an end part of each piece and the force applied to a part close to the intersection part 111 is not uniform. This problem is changeable depending on a distance between the sensors and a thickness of the biological tissue into which a travel axis of transmission and reception signals is inserted, in particular, since the both parts are fixed to the sensor and the measurement unit with the thickness of the oblique structure, this may cause a larger measurement error. In addition, an excessive force may be applied to a portion of the biological tissue inserted between the pivotal tongs to cause pain when worn for a long time for continuous measurement. In addition, noninvasive probes with a tongs structure for measuring biological signs such as oxygen saturation and blood glucose generally do not include a separate means capable of measuring a thickness of an inserted biological tissue.

Meanwhile, a probe 200b shown in the lower part of FIG. 10 is a probe of a different type of pivotal tongs structure that allows a more uniform pressure to be applied to each part of the biological tissue 10 such as an earlobe located between the first part 21 and the second part 22. The shapes of the first part 21 and the second part 22 rotatably coupled to a fixed point 133 may be modified to apply a more uniform pressure to the biological tissue 10 with a more relaxed slope than that of the probe 200a described above. However, since this structure is also fundamentally a structure in which the first part 21 and the second part 22 pivot around the fixed point 133, a slope with respect to the biological tissue 10 to be measured still exists.

The biological tissue inserted between the first part and the second part of the probe is generally an aggregate of inhomogeneous tissues and thus inevitably affects the measurement signal in a chaotic manner depending on the time of measurement or individual differences. In particular, signal detection capable of deriving a certain correlation with the concentration of a target material and a signal processing method therefor are very important even when an intensity of a signal detected by the probe using an optical method tends to be chaotic due to chaotic scattering according to inhomogeneity inside the biological tissue and the effect of temperature change.

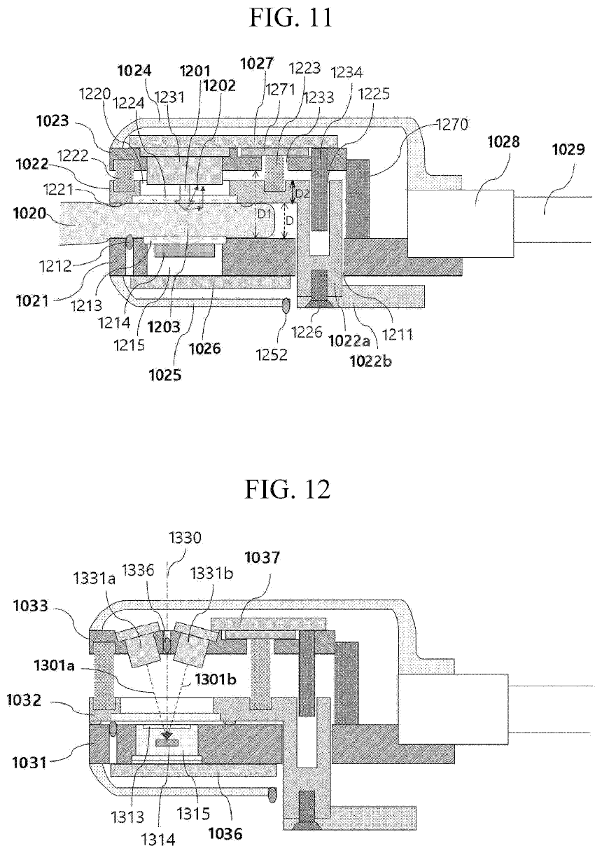

A probe structure of the present invention for overcoming such a limitation will be described in detail below with reference to the accompanying drawings. FIGS. 11 to 13 show a physical configuration of a balanced fastening-type probe, an element for measuring a thickness (or pressure) of biological tissue to be measured, and configurations of light sending and receiving elements for measuring light and/or photoacoustic transmission and light reflection according to a specific embodiment of the present invention.

First, FIG. 11 is a cross-sectional diagram illustrating an embodiment of a balanced-fastening-type probe for noninvasive blood glucose measurement according to the present invention.

Components of the balanced fastening-type probe fastened to biological tissue 1020 such as an earlobe largely include a lower frame 1021, a moving frame 1022, an upper frame 1023, an upper cover 1024, a lower cover 1025, a lower printed board assembly (PBA) 1026, an upper PBA 1027, a rubber boot 1028, and an electric wire interface 1029. The other components except for the moving frame 1022 are integrally assembled and have a fixed structure.

The upper frame 1023 and the lower frame 1021 are spaced apart from each other, and the upper frame 1023 and the lower frame 1021 are structurally fixed. In addition, the moving frame 1022 is located between the upper frame 1023 and the lower frame 1021 and forms a space separated from the lower frame 1021. The biological tissue 1020 is inserted into the separation space.

Since the moving frame 1022 is movable up and down between the upper frame 1023 and the lower frame 1021, the separation space is reduced or expanded by the movement of the moving frame 1022. Once the biological tissue 1020 is inserted into the separation space, the moving frame 1022 is pressed toward the biological tissue 1020 by an elastic force of elastic elements 1222 and 1223 such as compression spring, rubber, etc., and is brought into close contact with the moving frame 1022. When a user applies a force against the elastic force of the elastic elements 1222 and 1223, the space separated from the lower frame 1021 is widened and the biological tissue 1020 may be withdrawn or inserted.

Here, a separation space expansion mechanism is required for the user to apply a force against the elastic force of the elastic elements 1222 and 1223 to widen the space separated from the lower frame 1021. In order to configure the separation space expansion mechanism, the moving frame 1022 includes a pillar 1022a formed in an inverted “L” shape at a portion thereof. The pillar 1022a passes through a hole 1211 formed in the lower frame 1021, and is integrally coupled to a flat frame 1022b at a lower part of the lower frame 1021 by a screw 1226. As a result, the moving frame 1022 is assembled to receive a downward pressure by the elastic elements 1222 and 1223 between the lower frame 1021 and the upper frame 1023, which are integrally fixed and assembled, and a pillar 1234 serves as a guide for preventing the moving frame 1022 from being separated and inclined when the moving frame 1022 moves.

Since the flat frame 1022b integrally assembled to the lower part of the moving frame 1022 bent in the inverted “L” shape is exposed below the lower frame 1021, when a larger force is applied upward to the flat frame 1022b in a direction opposite to the elastic force of the elastic elements 1222 and 1223, the moving frame 1022 moves toward the upper frame 1023 and expands a separation distance (i.e., the separation space) from the lower frame 1021 to D so that the biological tissue 1020 such as an earlobe or flesh between fingers may be inserted into the separation space. A separation distance D1 between facing surfaces of the lower frame 1021 and the upper frame 1023 has a fixed value. Accordingly, in the moving frame 1022, when a thickness of the plate in contact with the biological tissue 1020 is D2, a maximum value of the separation distance D is designed to satisfy D(max)=D1−D2. Accordingly, a maximum thickness t of the biological tissue 1020 inserted into the probe is determined to satisfy t≤D(max).

Describing an operation sequence for inserting and fastening biological tissue, (1) press the flat frame 1022b with a hand to maximize D, (2) insert the biological tissue 1020 between the lower frame 1021 and the moving frame 1022, and then (3) remove a pressing force, so that the biological tissue 1020 is easily adhered and coupled between the moving frame 1022 and the lower frame 1021 by the elastic force of the elastic elements 1222 and 1223. Here, small protrusions 1221 additionally formed in the moving frame 1022 serve to prevent the biological tissue 1020 from being separated while being fastened to the probe.

In addition, the moving frame 1022 further includes a hole 1220 for a light signal path formed in accordance with a position of a light sending and receiving module 1231 assembled to the upper frame 1023, and a transparent window 1224 assembled to the hole 1220. The transparent window 1224 may be transparent glass or transparent plastic having a small thickness.

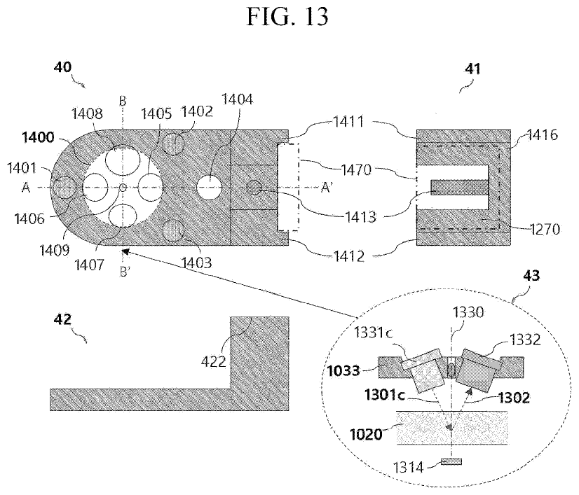

In the upper frame 1023, a bottom 1422 of both pillars 1411 and 1412, which are shown in a side view of FIG. 13 shown with different angle views, is assembled and coupled to the lower frame 1021, but additional parts such as screws for assembly are not shown.

The moving frame 1022 and the upper frame 1023 have additional structures such as grooves or holes into which the elastic elements 1222 and 1223 may be inserted and assembled. Alternatively, the upper frame 1023 is assembled by including a circular or polygonal pillar 1234, and the moving frame 1022 further includes a hole 1225 through which the pillar 1234 may move. As a result, the moving frame 1022 is assembled to receive a downward pressure by the elastic elements 1222 and 1223 between the lower frame 1021 and the upper frame 1023, which are integrally fixed and assembled, and the pillar 1234 serves as a guide for preventing the moving frame 1022 from being separated and inclined when the moving frame 1022 moves.

In addition, as shown in FIG. 11, in the balanced fastening-type probe according to the embodiment of the present invention, in order to monitor the coupling state with the biological tissue 1020, a pressure sensor 1271 and an elastic element 1223 are closely coupled between the moving frame 1022 and the upper PBA 1027 assembled on the upper part of the upper frame 1023. One end of the elastic element 1223 is fixed to the moving frame 1022, and the other end passes through a hole 1233 formed in the upper frame 1023 to be located at the center of the pressure sensor 1271 located below the upper PBA 1027. As a result, a difference in pressure applied to the pressure sensor 1271 is generated by the elastic element 1223 according to the coupling state between the probe and the biological tissue 1020, and thus whether the probe is coupled to the biological tissue and a thickness of the biological tissue may be monitored as described below in FIG. 17.

The upper frame 1023 includes the light sending and receiving module 1231 and the upper PBA 1027 connected to the light sending and receiving module 1231, and is fixedly assembled. The light sending and receiving module 1231 may include the light source units 27 and 28, the receiving units 23 and 24, and the sensor units 25 and 26 for measuring a temperature and a thickness, which are described above with reference to FIG. 2. The light source units 27 and 28 may be configured as two or more light sending elements such as LDs or LEDs, and the receiving unit 24 may be configured as a photodiode (PD) or an ultrasound detection element in order to receive a light transmission signal and/or an ultrasound signal. Light energy or light transmission signal 1201 output from the light sending element is incident on the biological tissue 1020 through the transparent window 1224, and a portion of incident light energy is reflected from a surface of the biological tissue and the remaining portion thereof is absorbed and scattered therein. The light energy scattered and reflected by organic molecules containing glucose and cells in the biological tissue but returned to an incident direction without being absorbed and dissipated is expressed as light reflection energy, and a portion 1202 of the light reflection energy in the biological tissue may be detected by the receiving unit of the light sending and receiving module 1231. Meanwhile, the light energy irradiated to the biological tissue from the light sending and receiving module 1231 may be a modulated signal for an ultrasound frequency, and in this case, the light energy absorbed in organic molecules such as glucose molecules or cells inside the biological tissue may be converted into heat or may generate an ultrasound signal 1203 by the photoacoustic effect.

Meanwhile, an ultrasound module 1215 and the lower PBA 1026 connected to the ultrasound module 1215 are assembled to the lower frame 1021. The ultrasound module 1215 may include an ultrasound element 1214 and an impedance matching plate 1213 for assisting smooth transmission of ultrasound to the ultrasound element 1214. At least a portion of the ultrasound signal 1203 generated in the biological tissue 1020 and passing through the biological tissue 1020 passes through the impedance matching plate 1213 and is detected by the ultrasound element 1214.

Unlike the conventional pivot-type probe described with reference to FIG. 10, the probe according to an embodiment of the present invention has a feature in which the lower frame 1021 and the upper frame 1023 have the constantly fixed separation distance D1 and do not move with respect to the biological tissue 1020. Accordingly, a separation distance between the light sending and receiving module 1231 and the ultrasound module 1215 coupled to the respective frames and a central axis of the signals transmitted therebetween may also be maintained in a constant state. Accordingly, it can be expected that unique characteristics of the biological tissue 1020 inserted into a space between the sensors in the upper frame 1023 and the sensors in the lower frame 1021 of the probe may be more stably inspected without change in a distance of the space and in a direction in which the signal is transmitted.

In addition, the probe of FIG. 11 may further include a temperature sensor for temperature monitoring provided for each component as follows. First, the upper frame 1023 may include a temperature sensor (a first temperature sensor) therein for monitoring the temperature of the light sending and receiving module 1231 as described above. Second, the lower frame 1021 includes a second temperature sensor 1212 assembled to partially protrude from a part being in contact with the biological tissue 1020, thereby monitoring a surface temperature of the biological tissue 1020. Temperature information of the second temperature sensor 1212 may also be used for monitoring a temperature of the ultrasound-receiving module 1215 adjacent thereto. A third temperature sensor 1252 is assembled to the lower cover 1025 and is used as a means for monitoring changes in the atmospheric temperature of a probe housing and therearound. All of these temperature sensors are electrically connected to the lower PBA 1026 and the upper PBA 1027.

In addition, the probe of FIG. 11 may further include a heating element 1270 and may use the heating element 1270 as a means capable of controlling the temperature of the upper frame 1023 and the lower frame 1021 of the probe. Since the biological tissue 1020 has a higher temperature than a room temperature, a temperature difference occurs when the biological tissue is coupled to the probe, and it takes some time until the temperature difference is minimized and the temperature of the probe reaches a temperature within a stable range. Accordingly, the heating element 1270 inside the probe may reduce a temperature stabilization time immediately after the probe is worn. In addition, even after the temperature of the probe is stabilized to be close to the temperature of the biological tissue 1020, the heating element 1270 may be used as an optional means for assisting the probe to be maintained within a reference temperature range (for example, a reference temperature of +/−1° C.).

A rubber boot 1028, which is another component not yet described in FIG. 11, allows the electric wire interface 1029 coming in from the outside to be fixed to the probe. The electric wire interface 1029 is electrically connected to the upper PBA 1027 and the lower PBA 1026, and is also connected to the bio-recorder 13.

FIG. 12 illustrates a cross-sectional structure of a probe according to another embodiment of the present invention. The light sending and receiving module 1231 and the ultrasound module 1215 described with reference to FIG. 11 may be configured by being changed to light modules 1331a and 1331b having multiple individual package types shown in FIG. 12 and light modules 1331c and 1332 shown in FIG. 13, a temperature sensor 1336, and a light-receiving module 1315. Accordingly, some structures of a lower frame 1031 and an upper frame 1033 for accommodating these changed elements must be changed. In addition, since other parts including a moving frame 1032 have the same function as those of FIG. 11, a description thereof will be omitted in FIG. 12. In addition, FIG. 12 illustrates an example of a state in which the biological tissue 1020 is not coupled.

In FIG. 12, a plurality (four in the examples of FIGS. 12 and 13) of individual light modules (1331a and 133b, and 1331c and 1332 shown in FIG. 13) are assembled to the upper frame 1033 so as to have inclination angles facing each other with respect to a central axis 1330, the separate temperature sensor 1336 is assembled at the center of the individual light modules, and the individual light modules are electrically connected to an upper PBA 1037. The plurality of individual light modules may include three light-transmitting modules (1331a and 1331b of FIGS. 12, and 1331c of FIG. 13) and one light-receiving module (1332 of FIG. 13), each of which includes an LD (or LED) for outputting light. In addition, the light-receiving module 1315 and a lower PBA 1036 electrically connected to the light-receiving module 1315 are assembled to the lower frame 1021. The light-receiving module 1315 may be composed of a light incident transparent window 1313, a light-receiving element 1314, and other packaging components.

A structure viewed from the above of the upper frame 1033 in FIG. 12 is illustrated in a plan view 40 of FIG. 13. In FIG. 12, the light-transmitting modules 1331a and 1331b installed in the upper frame 1033 represent two light modules located in a longitudinal axis (A-A′) shown in the plan view 40 of FIG. 13. The light-transmitting modules 1331a and 1331b are assembled to the upper frame 1033 such that central axes 1301a and 1301b of output light signals respectively emitted from of the light-transmitting modules 1331a and 1331b have an inclination so that the central axes 1301a and 1301b converge at the light-receiving element 1314 installed in the lower frame 1031. Meanwhile, since biological tissue (or bio-tissue) is an inhomogeneous object including epidermis, dermis, subcutaneous fat, muscle, blood and blood vessels including the same, interstitial fluid between cells, and the like, light energy incident to the inside is mostly absorbed and multi-scattered (or diffused). Photons multi-scattered by biological tissue particles having a size smaller than an incident wavelength tend to be backscattered in a direction opposite to an incident direction due to the Rayleigh scattering effect, and photons multi-scattered by particles having a size larger than the incident wavelength tend to be forward scattered in the traveling direction due to the Mie scattering effect. Accordingly, the purpose of aligning the central axes 1301a and 1301b of the output light signals to the light-receiving element 1314 is to increase the probability for stably receiving diffused transmission characteristics of the biological tissue by positioning the receiving element at a center portion of a multi-forward scattering photon cluster, rather than expecting a direct light transmission effect.

The other two of the four light modules assembled to the upper frame 1033 of FIG. 12 are installed along a transverse axis (B-B′) as in the plan view 40 of FIG. 13, and are assembled to the structures of the light-transmitting module 1331c and the light-receiving module 1332 shown in a dotted circle 43, which is a detailed view. These two light modules 1331c and 1332 are also assembled to have predetermined angles facing each other with respect to the central axis 1330 therebetween, and are preferably assembled such that the central axis 1301c of the light signal output from the light-transmitting module 1331c and the central axis 1302 of the light signal received by the light-receiving module 1332 face the middle depth of the biological tissue 1020. Accordingly, a portion of the light energy output from the light-transmitting module 1331c is reflected and scattered from the biological tissue 1020 and is easily incident on the light-receiving module 1332.

In order to help understanding the overall structure of the upper frames 1023 and 1033 and to describe additional functions, FIG. 13 illustrates the plan view 40 illustrating the upper frames 1023 and 1033, a first side view 41 viewed from a side indicated by A′, and a second side view 42 viewed from a side indicated by B′. In particular, in order to describe all of the upper frames 1023 and 1033, some structures are illustrated by overlapping each other in the plan view 40, and the upper frames 1023 and 1033 shown in FIGS. 11 and 12 correspond to cross-sectional structures in a direction of the longitudinal axis (A-A′) of FIG. 13. In the plan view 40, a dotted circle 1400 indicates a position in which the light sending and receiving module 1231 is installed in the upper frame 1023 of FIG. 11, and five solid circles 1405, 1406, 1407, 1408, and 1409 arranged along the dotted circle 1400 indicate positions in which the four light modules 1331a, 1331b, 1331c, and 1332 and the temperature sensor 1336 described above are installed, respectively.

In addition, in the upper frames 1023 and 1033, three circular grooves 1401, 1402, and 1403 are formed with a diameter and a certain depth that can accommodate the above-described elastic elements, and a through hole 1404 is formed to accommodate another elastic element described above. Specifically, the groove 1401 is a position at which the elastic element 1222 of FIG. 11 is assembled, and the groove 1404 corresponds to the hole 1233 described in FIG. 11 and indicates a position through which the elastic element 1223 passes. The grooves 1402 and 1403 are not shown in FIGS. 11 and 12, but other elastic elements may be accommodated in the grooves 1402 and 1403. That is, the elastic elements in the probe according to an embodiment of the present invention may be configured with only two elastic elements accommodated in the positions of the groove 1401 and the hole 1404, and may be configured to have four elastic elements by additionally including two elastic elements accommodated in the grooves 1402 and 1403.

As shown in the first side view 41 of FIG. 13, two protrusions 1411 and 1412 formed at one ends of the upper frames 1023 and 1033 are assembled while one surface 1422 seen in the second side view 42 comes into contact with the lower frame 1021 of FIG. 11. In the drawing, screws, threads, and the like required for assembly are omitted for simplicity. In addition, a pillar 1413 represents the pillar 1234 described in FIG. 11, and a region 1470 shown by a one-dot chain line indicates a position at which the heating element 1270 of FIG. 11 is closely assembled to the upper frame 1023 or 1033.

As described above, the balanced fastening structure probe for noninvasive blood glucose measurement according to the present invention basically provides a means capable of inserting and fixing biological tissues such as earlobes of various thicknesses between frames in a state in which a transmitting element and a receiving element are always installed in a constant form on the frames of the probe. In addition, although the light sending and receiving elements and the ultrasound element are mainly described in FIGS. 11 to 13, the frame of the probe according to the present invention may accommodate various types of transmitting and receiving elements.

As an example, in FIG. 11, the light sending and receiving module 1231 may be used in place of optical collimators coupled to an optical fiber to generate parallel light. In addition, these collimators may be coupled to multi-mode optical fibers to be connected to halogen lamps and spectrometer measurement apparatuses.

Hereinafter, an operation of the noninvasive blood glucose measurement apparatus and method, measurement examples of the light reflection and the transmission, and signal processing methods will be described.

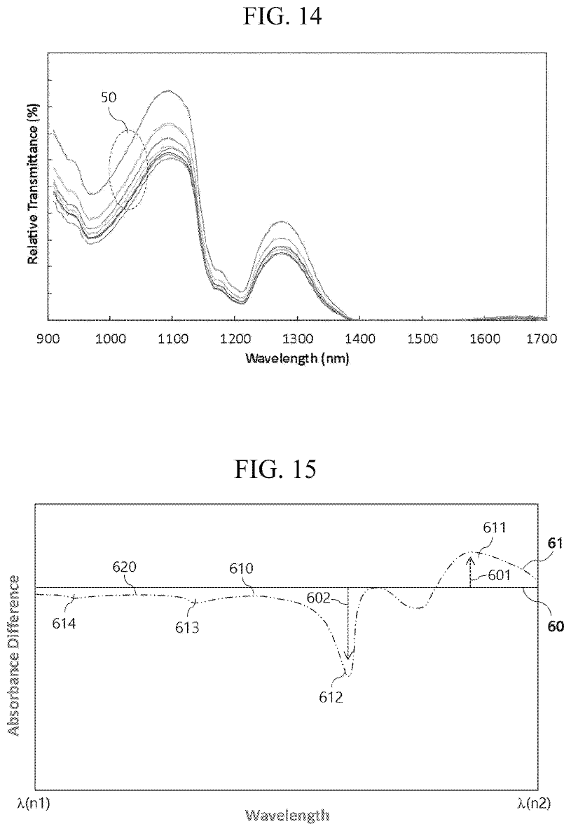

FIG. 14 shows characteristics 50 obtained by measuring a transmission spectrum of an earlobe by applying the probe according to the present invention. In FIG. 14, transmission spectrum measurement signals 50 for an earlobe of the human body show different transmittance characteristics, which reflect a difference in the characteristics of a spring fastened to a probe platform, thickness and surface reflection according to a position of the earlobe, and the like. When the measurement is performed by changing the type of elastic element coupled to the probe, a difference in transmission characteristics may occur due to a difference in the thickness of the earlobe inserted into the probe because the magnitude of a pressure applied to the biological tissue of the earlobe is different according to a difference in elasticity. In addition, since the thickness is not constant depending on the position on the earlobe due to the characteristics of the human body, and there may be differences in skin color, hair, dots, and internal tissues, this also causes a difference in transmittance. Even in the biological tissue to be measured which is considered to be the same, as described above, slight differences occur in actual thickness, temperature, reflection and transmission characteristics of transmission signals, and the like during repeated noninvasive measurements. Accordingly, it is desirable that the probe further includes a means capable of measuring thickness and temperature together with a reproducible transmission and reception measurement method for accurate measurement and correction.

FIG. 15 is a graph for describing an example of signal processing of an absorbance difference spectrum according to a glucose concentration. That is, a difference curve (two-dot chain line 61) between an absorbance according to light transmission when the glucose concentration is 0 mg/dL and an absorbance when the glucose concentration is N (>0) mg/dL in a near-infrared wavelength region of λ(n1) to λ(n2) is shown, and a solid line 60 indicates the case in which the difference in absorbance is zero when the glucose concentration is 0 mg/dL. An arrow 601 indicates that the absorbance gradually increases in a wavelength region (for example, 611) in which the curved line 61 is higher than the straight line 60, together with an increase in glucose concentration, and an arrow 602 means that the absorbance gradually decreases in a wavelength region (for example, 612 to 614) in which the curved line 61 is lower than the straight line 60. In the curved line 61, a wavelength region in which absorbance reactivity according to an increase in glucose concentration is the greatest is divided into a valley 612 (absorbance difference in a negative direction), followed by a crest 611 (absorbance difference in a positive direction), a valley 613, and a valley 614 in order. In addition, in the curved line 61, 610 and 620 mean a wavelength region in which absorbance reactivity according to an increase in glucose concentration is the smallest. By configuring wavelength channels for the light-sending element of the probe according to the present invention on the basis of the absorbance reactivity according to the glucose concentration, the noninvasive blood glucose measurement can be continuously performed. That is, a plurality of wavelength channels are selectively applied to the sent light signal 1201 and the light sending and receiving module 1231 of FIG. 11, and the sent light signal and the light sending and receiving modules 1331 and 1332 of FIG. 12 according to the degree of the glucose reactivity described in FIG. 15.

First, as a step-wise process for the blood glucose measurement, a transmission signal for a light wavelength λg targeted in a wavelength region (for example, the wavelength region 612 of FIG. 15) having the highest reactivity to a glucose solution is selected.

Next, at least one transmission signal of an arbitrary comparison wavelength λc of light for relative correction is selected in wavelength regions (for example, 610, 620, 613, and 614 in FIG. 15) having no glucose reactivity or low reactivity.

The selected multi-channel light signals are sent through the biological tissue to be measured, and an intensity of a reception signal sent at each wavelength is measured, and a wavelength ratio of λc/λg is obtained by dividing an intensity of a reception signal transmitted at the comparison wavelength λc by an intensity of a reception signal transmitted at a target wavelength λg. The wavelength ratio of λc/λg is measured in this way for each of samples having different glucose concentrations, and a relative difference (defined as a relative ratio) for the sample with the glucose concentration of 0 mg/dL is finally obtained. The above description may be more clearly expressed using equations. That is, a ratio R(λ_{g}^{c}), at the two wavelengths of λc/λg, of an intensity I(λ_{c}) of the reception signal transmitted at the comparison wavelength λc described in FIG. 15 to an intensity I(λ_{g}) of the reception signal transmitted at the target wavelength λg are expressed by Equation (1) below.

R(λ_{g}^{c})=I(λ_{c})/I(λ_{g}) (1)

In addition, a relative difference Dᵣ^{s}(λ_{g}^{c}) between the ratio Rᵣ(λ_{g}^{c}) at the wavelengths of λc/λg in the measurement sample and the ratio Rₛ(λ_{g}^{c}) at the wavelengths of λc/λg in the reference sample in which the glucose concentration is 0 mg/dL is summarized as an equation and is expressed as Equation (2) below.

$${D}_{r}^{s} ( {λ}_{g}^{c} ) = {R}_{s} ( {λ}_{g}^{c} ) / {R}_{r} ( {λ}_{g}^{c} ) = \frac{{I}_{s} ( {λ}_{c} ) / {I}_{s} ( {λ}_{g} )}{{I}_{r} ( {λ}_{c} ) / {I}_{r} ( {λ}_{g} )} \qquad ( 2 )$$

Next, pieces of data obtained by measuring various relative ratios according to actual glucose concentrations in the biological tissue to be measured are accumulated, and a standard comparison table may be created based on the accumulated data. The actual glucose concentration for the biological tissue may be utilized on the basis of a value measured through a conventional blood drawing or minimally invasive blood glucose meter. A reference comparison table prepared by extracting learning data for noninvasive blood glucose measurement using the above method is input to the bio-recorder 13 or the like.

Now, the biological tissue to be measured in a state in which a blood glucose value is unknown is inserted into the probe, and a relative ratio is measured from the target wavelength kg and the comparison wavelength λc described above, and a blood-glucose concentration value corresponding to the measured relative ratio is found from the reference comparison table and displayed as the evaluated blood glucose level. Since a glucose concentration in the human body may not be 0 mg/dL, a reference value in the reference comparison table having a blood glucose value of a predetermined level extracted from the learning data described above may be used instead of the reference sample having a glucose concentration of 0 mg/dL as described with reference to FIG. 15 and Equation (2).

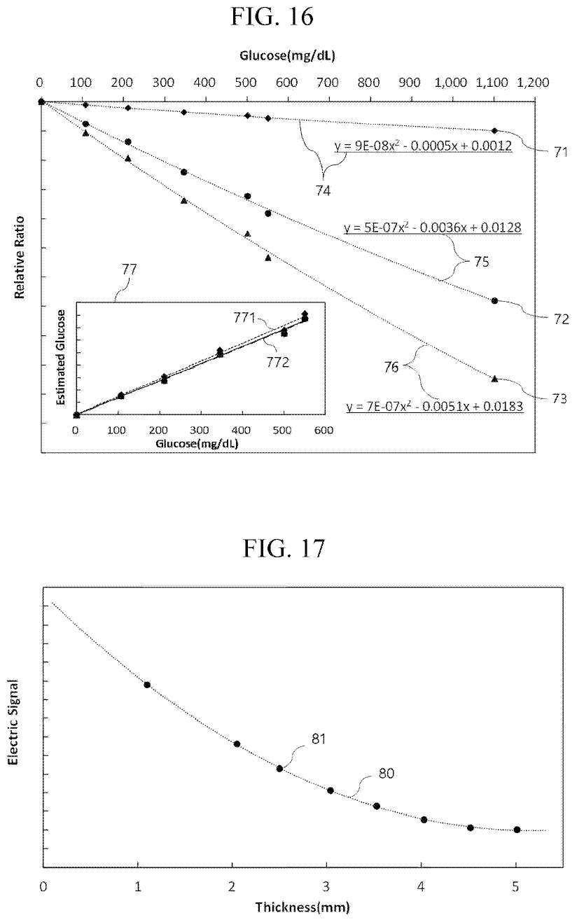

FIG. 16 shows an example of a result of measuring samples in which glucose concentrations range from 0 to 1100 mg/dL by the noninvasive blood glucose measurement method using the above-described wavelength channel assignment method. Results of measuring relative ratios of the three comparison wavelengths λc, which are respectively named as λ1, λ2, and λ3, to the selected one target wavelength λg are shown.

Specifically, the target wavelength λg is in the wavelength region 612 of FIG. 15, and the comparison wavelengths λ1, λ2, and λ3 may be selected from the wavelength regions 611, 613 (or 614), 610 (or 620), respectively, in FIG. 15. In FIG. 16, Graph 71 shows relative ratio measurement data for λ1/λg, and Graphs 72 and 73 show relative ratio measurement data for λ2/λg and λ3/λg, respectively. In FIG. 16, dotted lines and Equations 74, 75, and 76 indicate quadratic/polynomial trendlines for pieces of data 71, 72, and 73 and equations thereof, respectively. The difference in size of the relative ratio characteristic in Graphs 71 to 73 is due to the difference in transmittance at each comparative wavelength, and the relative ratio according to the glucose concentration is higher at the comparative wavelength λ3 having relatively higher transmittance than the comparative wavelengths λ1 and λ2 having small transmittance, and thus it is more advantageous in signal-to-noise ratio. However, when there is no significant difference in the signal-to-noise ratio between the comparison wavelengths with the sufficient transmission signal intensity, normalized glucose evaluation values with respect to the maximum measured value show almost the same results. That is, Graph 77 inserted in a lower end of FIG. 16 indicates a glucose concentration that is noninvasively evaluated with respect to the glucose concentration of the actual sample. Graph 771 of a dotted line is obtained by normalizing the data 71, and Graph 772 is obtained by normalizing the pieces of data 72 and 73 in which the signal-to-noise ratio characteristic is excellent, which show almost overlapping characteristics.

Meanwhile, the above-described basic signal processing characteristics are maintained to be the same, but the relative ratio Dᵣ^{s}(λ_{g}^{c}) in Equation (2) is used in a rearranged form as in Equation (3) by adding a natural logarithm and a minus (−) sign according to the convenience of use, thereby increasing the visibility of the characteristic analysis as necessary,

$$- ln ( {D}_{r}^{s} ( {λ}_{g}^{c} ) ) = - ln ( \frac{{I}_{s} ( {λ}_{c} )}{{I}_{r} ( {λ}_{c} )} ) + ln ( \frac{{I}_{s} ( {λ}_{g} )}{{I}_{r} ( {λ}_{g} )} ) = {E}_{c} ( {λ}_{c} ) - {E}_{c} ( {λ}_{g} ) ≡ Δ {E}_{c} ( {λ}_{g}^{c} ) \qquad ( 3 )$$

In Equation (3),

$$( \frac{{I}_{s} ( {λ}_{c} )}{{I}_{r} ( {λ}_{c} )} )$$

is transmittance of the measurement sample relative to the reference sample at the comparison wavelength λc, and −ln( ) for the transmittance means comparative extinction with respect to the reference sample at the comparative wavelength λc, and thus may be expressed as

$${E}_{c} ( {λ}_{c} ) = - ln ( \frac{{I}_{s} ( {λ}_{c} )}{{I}_{r} ( {λ}_{c} )} ) .$$

### Likewise,

$${E}_{c} ( {λ}_{g} ) = - ln ( \frac{{I}_{s} ( {λ}_{g} )}{{I}_{r} ( {λ}_{g} )} )$$

means comparative extinction of the measurement sample with respect to the reference sample at the target wavelength λg. Physically, extinction indicates a degree by which light energy incident on the biological tissue is absorbed and scattered internally and attenuated, and comparative extinction relative to the reference sample is a term expressing a degree of relative extinction to the reference sample.

Equation (3) may be defined as a difference of the extinction of the measurement sample relative to the reference sample at each of the comparison wavelength λc and the target wavelength χg, that is ΔE_{c}(λ_{g}^{c}), and may be named and expressed as a normalized extinction difference (NED) method. The NED method implicitly represents that a difference in extinction appears when the natural logarithm and minus calculations are applied on the result reflecting both the normalization between the reference sample and the measurement sample and the normalization between the comparison wavelength λc and the target wavelength λg.

Such a signal processing method of the present invention provides an efficient means for deriving a correlation with respect to a target material concentration from chaotic measurement signals that are diffusely transmitted through or reflected by an inhomogeneous material such as biological tissue.

Meanwhile, the above-described balanced fastening probe according to an embodiment of the present invention includes a means for measuring a thickness of an inserted biological tissue by effectively using spatial structural characteristics between the moving frames 1022 and 1032 and the lower frames 1021 and 1031. That is, the thickness of the biological tissue may be estimated from an electrical signal such as a resistance or a voltage output from the pressure sensor 1271 through the means 1022, 1023, 1027, 1271, and 1223 related to the thickness measurement based on the pressure applied to the biological tissue described in FIG. 11. As the thickness of the biological tissue inserted between the moving frames 1022 and 1032 and the lower frames 1021 and 1031 of the probe increases, the length of the elastic element becomes shorter, and thus the pressure applied to the pressure sensor 1271 located at one end of the elastic element increases, and conversely, as the thickness decreases, the pressure decreases. FIG. 17 shows an electrical resistance characteristic graph 80 measured for biological tissues having a thickness in the range of, for example, 1 to 5 mm. When a unique thickness-electrical signal characteristic is measured using the thickness measurement means fixed to the probe as described above, and an electrical signal value is measured as in a point 81 for each thickness of the biological tissue on the basis of the thickness-electrical signal characteristic, the value converted to each thickness value of the biological tissue inserted into the probe may be monitored in real time.

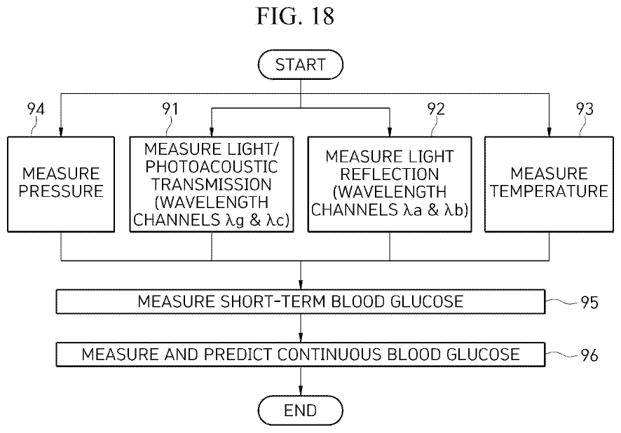

FIG. 18 illustrates a noninvasive continuous blood glucose measuring method according to another embodiment of the present invention as a flowchart. In order to further clarify the description of FIG. 18, first, the concepts of measurement of the light reflection, the light transmission and/or photoacoustic transmission described with reference to FIGS. 11 and 12 are summarized together as follows.

The light sending and receiving module 1231 included in the upper frame 1023 of FIG. 11 and the plurality of light modules 1331a, 1331b, 1331c, and 1332 and the temperature sensor 1336 included in the upper frame 1033 of FIG. 12 may be used interchangeably, and the basic unique functions for outputting multi-wavelength light signals, receiving light reflection signals, and monitoring temperature are the same (however, there is a difference in paths through which the output light signals are incident on the biological tissue due to the difference in the arrangement structure, and slightly different effects may be generated due to the difference in the paths of the transmitted and reflected signals). Accordingly, (1) the measurement of the light reflection made on the upper frames 1022 and 1033 is expressed by measuring light reflection on both upper frames despite the difference in the detailed path. The ultrasound module 1215 included in the lower frame 1021 of FIG. 11 receives a photoacoustic transmission signal 1203 that is generated inside the biological tissue by the light signals output from the upper frame. Meanwhile, the light-receiving module 1315 included in the lower frame 1031 of FIG. 12 may receive light transmission signals 1301a, 1301b, and 1301c generated by the light signals output from the upper frame and diffused and transmitted inside the biological tissue. As a result, it is assumed that these two different transmission signals (the photoacoustic transmission signal and the light transmission signal) are both or selectively (alternatively) applied to the probe according to the present invention. Accordingly, (2) one or both of these two methods are selectively used in the light/photoacoustic transmission measurement performed in the lower frames 1021 and 1031.

The process shown in the flowchart of FIG. 18 is described with a case of a combination including a light reflection measurement 92, a light transmission measurement 91, a temperature measurement 93, and a pressure measurement 94, and a similar process may also be applied in a case of a combination including a photoacoustic transmission measurement instead of or in addition to the light transmission measurement 91. In addition to the light reflecting element as described in FIGS. 11 and 12, bio-signal measurement signals may be monitored and collected by using a probe to which the temperature and thickness measurement means is additionally coupled.

In the light and/or photoacoustic transmission measurement 91, the output light signals 1201, 1301a, 1301b, and 1301c output from the upper frames 1023 and 1033 described in FIGS. 11 and 12 are scattered and transmitted in the biological tissue and/or generated as photoacoustic signals, the signals transmitted to the light-receiving module 1315 and/or the ultrasound module 1215 of the lower frame 1031 are detected, and light and/or photoacoustic transmission characteristics are measured using a result of the relative ratio Dᵣ^{s}(λ_{g}^{c}) or the normalized extinction difference ΔE_{c}(λ_{g}^{c}) described in FIGS. 15 and 16.

In the light reflection measurement 92, the output light signals 1201, 1301a, 1301b, and 1301c output from the upper frames 1023 and 1033, diffused and reflected in the biological tissue, and transmitted to the light sending and receiving module 1231 or the light module 1332 of the upper frame are detected, and the light reflection characteristics are measured using the relative ratio Dᵣ^{s}(λ_{g}^{c}) or the normalized extinction difference ΔE_{c}(λ_{g}^{c}) described in FIGS. 15 and 16.

In the temperature measurement 93, multiple pieces of temperature information are simultaneously measured based on a first temperature sensor (e.g., 1336, or included in 1231) included in the upper frames 1023 and 1033 for monitoring the temperature of the light modules, of, and a second temperature sensor 1212 for monitoring the temperature on the surface of the biological tissue or in the vicinity of a bio-signal receiving module, and a third temperature sensor 1252 for monitoring the temperature of the housing of the probe or ambient air temperature.

In the pressure measurement 94 a pressure signal is detected through an organic interaction of the thickness measurement means (1022, 1223, 1027, 1271, and the like) included in the upper frames 1023 and 1033 to use as information for determining whether biological tissue is inserted into the probe, and produce a measurement result monitored in real time up to a change in the thickness of the inserted biological tissue on the basis of the pressure signal as shown in the method of FIG. 17.

A short-term blood glucose value measurement 95 includes (1) a process of deriving an average value by repeating the light and/or photoacoustic transmission measurement 91, the light reflection measurement 92, the temperature measurement 93, and the pressure measurement 94 N times during a certain time interval Δt, (2) a process of correcting the light transmission measurement and light reflection measurement results on the basis of the temperature and pressure measurement values, (3) a process of deriving each expected blood glucose level by comparing and analyzing the reference comparison table obtained in advance through prior learning data, the averaged and derived light and/or photoacoustic transmission measurement result, and the light reflection measurement results, (4) a process of finally determining a short-term blood glucose level on the basis of the blood glucose estimation values derived from the light and/or photoacoustic transmission and light reflection measurements, and 5) a process of recording and outputting the finally determined short-term blood glucose value. As an example, the short-term blood glucose value may be measured through repeated measurement and correction for a period of several seconds to several minutes.

A continuous blood glucose measurement and prediction 96 includes a process of repeatedly performing the short-term blood glucose value measurement 95 for a predetermined period of time (e.g., 5 minutes) and recording and outputting pieces of blood glucose value data (12 pieces of data per hour, 288 pieces of data in 24 hours) accumulated for a long time (e.g., 24 hours). Furthermore, the continuous blood glucose measurement and prediction 96 may further include a process of predicting an expected blood glucose value after predetermined lengths of time on the basis of the data accumulated and measured for the predetermined period of time or more.

The probe for noninvasive blood glucose measurement according to the embodiment described above may equally be used for round-shaped biological tissues including bones, such as fingers and toes, as well as thin biological tissues at the extremities of the human body, such as earlobes and flesh between the fingers. For example, the components constituting the probe of the new structure according to the present invention may be equally applied by manufacturing and applying the part of the probe that is closely applied to biological tissue in a flat or round shape.

According to the present invention, a noninvasive blood glucose measurement apparatus and method using multiple sensors can be provided in which a blood-glucose concentration for a biological tissue part such as an earlobe of the human body can be measured. More stable and accurate signal measurement is possible by always maintaining an interval and directionality of signal measurement components constant although the biological tissue varies in thickness. It is also possible to automatically and simultaneously perform personalized thickness correction in noninvasive measurement of a bio-signal by additionally including a means for measuring a thickness of the biological tissue inserted into a probe.

In addition, since the noninvasive blood glucose measurement apparatus and method according to embodiments of the present invention use a light source in a near-infrared wavelength region, manufacturing costs can be reduced, and miniaturization is possible so that it can be easily carried.

A human body wearable probe can be worn more conveniently even when the measurement is performed continuously for a long time, by applying a uniform force (pressure) to the human body on the basis of a probe platform of a new balanced fastening-type tongs structure to solve the problems of a conventional scissors type or pivotal tongs structure.

In the above, the embodiments specifically implementing the spirit of the present invention have been described. However, the technical scope of the present invention is not limited to the above-described embodiments and drawings, but is determined by reasonable interpretation of the claims.

### DESCRIPTION OF MAJOR COMPONENTS WHOSE NAMES ARE NOT SHOWN IN THE DRAWINGS

10: earlobe, 11: measurement region, 12: data communication element, 13: bio-recorder, 14: finger, 200: noninvasive probe worn on human body, 1021 and 1031: lower frame, 1022 and 1032: moving frame, 1023 and 1033: upper frame, 1212, 1252, and 1336: temperature sensors, 1215: ultrasound module, 1222 and 1223: elastic element, 1231: light sending and receiving module, 1234 and 1413: pillars, 1270: heating element, 1271: pressure sensor, 1331a, 1331b, 1331c, and 1332: light modules, 1315: light-receiving module

What is claimed is:

1. A noninvasive blood glucose measurement apparatus comprising:
a first light source unit disposed in a probe and configured to irradiate light of at least one wavelength to a biological tissue;
a second light source unit disposed in the probe and configured to irradiate light of at least one wavelength, which is different from that of the light of the first light source unit, to the biological tissue;
a first receiving unit disposed in the probe and configured to receive a transmission signal generated as the light irradiated to the biological tissue passes through the biological tissue;
a second receiving unit configured to receive a light reflection signal generated as the light irradiated to the biological tissue is reflected by the biological tissue; and
a measurement unit configured to measure light reflection characteristics of the biological tissue by using the light reflection signal received by the second receiving unit, measure transmission characteristics of the biological tissue by using the transmission signal received by the first receiving unit, and measure blood glucose of the biological tissue on the basis of the light reflection characteristics and the transmission characteristics.

2. The noninvasive blood glucose measurement apparatus of claim 1, wherein the transmission signal received by the first receiving unit is a photoacoustic transmission signal, and
the measurement unit is configured to measure photoacoustic transmission characteristics of the biological tissue by using the photoacoustic transmission signal received by the first receiving unit.

3. The noninvasive blood glucose measurement apparatus of claim 1, wherein the transmission signal received by the first receiving unit is a light transmission signal, and
the measurement unit is configured to measure light transmission characteristics of the biological tissue by using the light transmission signal received by the first receiving unit.

4. The noninvasive blood glucose measurement apparatus of claim 1, wherein the second light source unit irradiates light of a first wavelength channel, and light of a second wavelength channel having a relatively higher reactivity to glucose than the first wavelength channel, and
the first light source unit irradiates light of a third wavelength channel, and light of a fourth wavelength channel having a relatively higher reactivity to glucose than the third wavelength channel.

5. The noninvasive blood glucose measurement apparatus of claim 4, wherein the measurement unit measures the light reflection characteristics of the biological tissue by combining a light reflection signal for the first wavelength channel and a light reflection signal for the second wavelength channel or the fourth wavelength channel.

6. The noninvasive blood glucose measurement apparatus of claim 4, wherein the measurement unit measures the transmission characteristics of the biological tissue by combining a transmission signal for the third wavelength channel and a transmission signal for the second wavelength channel or the fourth wavelength channel.

7. The noninvasive blood glucose measurement apparatus of claim 1, further comprising a first sensor unit configured to measure a temperature of the biological tissue,
wherein the measurement unit measures the blood glucose of the biological tissue by using the temperature of the biological tissue.

8. The noninvasive blood glucose measurement apparatus of claim 7, further comprising:
a temperature sensor configured to monitor a temperature of at least one of the first light source unit, the second light source unit, the first receiving unit, and the second receiving unit; and
a temperature sensor configured to monitor a change in ambient air temperature of the probe.

9. The noninvasive blood glucose measurement apparatus of claim 1, further comprising a second sensor unit configured to measure a thickness of the biological tissue,
wherein the measurement unit measures the blood glucose of the biological tissue by using the thickness of the biological tissue.

10. The noninvasive blood glucose measurement apparatus of claim 9, wherein the second sensor unit includes:
a light-sending element disposed at a position of a first part of the probe, which is spaced apart from the biological tissue;
two or more light-receiving elements disposed at different positions of the first part of the probe, which are spaced apart from the biological tissue; and
a reflecting element disposed at a position of a second part of the probe, which is spaced apart from the biological tissue, and
the measurement unit is configured to measure the thickness of the biological tissue by measuring an intensity difference or ratio of light signals generated as light irradiated from the light-sending element is reflected by the reflecting element and then received by the two or more light-receiving elements.

11. The noninvasive blood glucose measurement apparatus of claim 9, wherein the second sensor unit includes a pressure sensor configured to measure a pressure applied by the probe to the biological tissue and output the measured pressure as an electrical signal, and
the measurement unit is configured to estimate the thickness of the biological tissue by using the electrical signal output from the pressure sensor.

12. The noninvasive blood glucose measurement apparatus of claim 1, wherein the second light source unit includes a light source element configured to irradiate light of different inclination angles to the biological tissue, and
the second receiving unit includes a receiving element configured to receive the light reflection signal at different inclination angles at a position corresponding to the light source element.

13. The noninvasive blood glucose measurement apparatus of claim 1, wherein the measurement unit measures the light reflection characteristics and the transmission characteristics of the biological tissue by distinguishing a wavelength region for measuring the light reflection characteristics and a wavelength region for measuring the transmission characteristics, and selectively applying at least one wavelength channel having a relatively low reactivity to glucose and at least one wavelength channel having a relatively high reactivity to glucose in the distinguished wavelength regions.

14. The noninvasive blood glucose measurement apparatus of claim 1, wherein the probe includes:
an upper frame;
a lower frame spaced apart from and fixed to the upper frame; and
a moving frame that is located between the upper frame and the lower frame, has a space separated from the lower frame, into which the biological tissue is inserted, and moves between the upper frame and the lower frame so that the space separated from the lower frame is changed,
wherein the moving frame is configured to apply a pressure to the biological tissue by an elastic element placed between the moving frame and the upper frame.

15. The noninvasive blood glucose measurement apparatus of claim 14, wherein the moving frame further includes a separation space expansion mechanism configured to widen the space separated from the lower frame when a force is applied against the elastic element.

16. The noninvasive blood glucose measurement apparatus of claim 14, wherein the first light source unit, the second light source unit, and the second receiving unit are installed in the upper frame, and
the first receiving unit is installed in the lower frame.

17. A noninvasive blood glucose measurement method comprising:
irradiating light of one or more different wavelengths to a biological tissue;
receiving a light reflection signal generated as the light irradiated to the biological tissue is reflected by the biological tissue;
receiving a transmission signal generated as the light irradiated to the biological tissue passes through the biological tissue; and
measuring light reflection characteristics of the biological tissue by using the light reflection signal, measuring transmission characteristics of the biological tissue by using the received transmission signal, and estimating blood glucose of the biological tissue on the basis of the light reflection characteristics and the transmission characteristics,
wherein the transmission signal includes at least one of a photoacoustic transmission signal and a light transmission signal, and
the transmission characteristics include at least one of photoacoustic transmission characteristics and light transmission characteristics.

18. The noninvasive blood glucose measurement method of claim 17, further comprising measuring at least one of a temperature and a thickness of the biological tissue.

19. The noninvasive blood glucose measurement method of claim 17, wherein the estimating of the blood glucose includes:
deriving an average value by repeating measuring the light reflection characteristics and measuring the transmission characteristics multiple times; and
deriving a blood glucose value by comparing the average value with a reference comparison table obtained in advance through prior learning data.

20. The noninvasive blood glucose measurement method of claim 19, further comprising repeatedly performing the deriving of the blood glucose value to output accumulated blood glucose value data for a predetermined period of time.
